(19) United States
(12) Patent Application Publication
(10) Pub. No.: US 2023/0321042 A1
(43) Pub. Date: Oct. 12, 2023

(54) COMBINATION THERAPY

(71) Applicant: PFIZER INC., NEW YORK, NY (US)

(72) Inventors: Stephen George DANN, Poway, CA (US); Nichol Lee Goodman MILLER, Poway, CA (US); Todd Lee VANARSDALE, San Diego, CA (US)

(73) Assignee: PFIZER INC., NEW YORK, NY (US)

(21) Appl. No.: 18/017,071

(22) PCT Filed: Jul. 16, 2021
(86) PCT No.: PCT/IB2021/056459

§ 371 Date: Jan. 19, 2023

Related U.S. Application Data
(60) Provisional application No. 63/054,016, filed on Jul. 20, 2020.

(51) Int. Cl. A61K 31/415; A61K 31/565; A61P 35/00; A61K 31/4196; A61K 31/519

(52) U.S. Cl. CPC A61K 31/415; A61K 31/4196; A61K 31/519; A61K 31/565; A61P 35/00

(57) ABSTRACT

This invention relates to combination therapies for use in treating cancer, comprising a cyclin dependent kinase 2 (CDK2) inhibitor of Formula (I), as further described herein, and a cyclin dependent kinase 4/6 (CDK4/6) inhibitor, optionally in further combination with an additional anti-cancer agent=.

### REFERENCE TO SEQUENCE LISTING

This application is being filed electronically via EFS-Web and includes an electronically submitted sequence listing in .txt format. The .txt file contains a sequence listing entitled “PC072650ASEQLISTING_ST25.txt” created on Jul. 7, 2021 and having a size of 1 KB. The sequence listing contained in this .txt file is part of the specification and is herein incorporated by reference in its entirety.

### BACKGROUND OF THE INVENTION

### Field of the Invention

The present invention relates to combination therapies useful for treating cancer. In particular, the invention relates to combination therapies comprising a cyclin dependent kinase 2 (CDK2) inhibitor of Formula (I), as further described herein and in U.S. Pat. No. 11,014,911, and a cyclin dependent kinase 4/6 (CDK4/6) inhibitor, optionally in further combination with an additional anti-cancer agent. The invention also relates to associated methods of treatment, pharmaceutical compositions, and pharmaceutical uses.

### Description of the Related Art

Cyclin-dependent kinases (CDKs) and related serine/threonine protein kinases are important cellular enzymes that perform essential functions in regulating cell division and proliferation. CDKs 1-4, 6, 10, 11 have been reported to play a direct role in cell cycle progression, while CDKs 3, 5 and 7-9 may play an indirect role (e.g., through activation of other CDKs, regulation of transcription or neuronal functions). The CDK catalytic units are activated by binding to regulatory subunits, known as cyclins, followed by phosphorylation. Cyclins can be divided into four general classes (G₁, G₁/S, S and M cyclins) whose expression levels vary at different points in the cell cycle. Cyclin B/CDK1, cyclin A/CDK2, cyclin E/CDK2, cyclin D/CDK4, cyclin D/CDK6, and likely other heterodynes are important regulators of cell cycle progression.

Overexpression of CDK2 is associated with abnormal regulation of the cell-cycle. The cyclin E/CDK2 complex plays and important role in regulation of the G1/S transition, histone biosynthesis and centrosome duplication. Progressive phosphorylation of retinoblastoma (RB) by cyclin D/Cdk4/6 and cyclin E/Cdk2 releases the G1 transcription factor, E2F, and promotes S-phase entry. Activation of cyclin A/CDK2 during early S-phase promotes phosphorylation of endogenous substrates that permit DNA replication and inactivation of E2F, for S-phase completion. (Asghar et al. The history and future of targeting cyclin-dependent kinases in cancer therapy, Nat. Rev. Drug. Discov. 2015; 14(2): 130-146).

Cyclin E, the regulatory cyclin for CDK2, is frequently overexpressed in cancer. Cyclin E amplification or overexpression has long been associated with poor outcomes in breast cancer. (Keyomarsi et al., Cyclin E and survival in patients with breast cancer. N Engl J Med. (2002) 347:1566-75). Cyclin E2 (CCNE2) overexpression is associated with endocrine resistance in breast cancer cells and CDK2 inhibition has been reported to restore sensitivity to tamoxifen or CDK4 inhibitors in tamoxifen-resistant and CCNE2 overexpressing cells. (Caldon et al., Cyclin E2 overexpression is associated with endocrine resistance but not insensitivity to CDK2 inhibition in human breast cancer cells. Mol. Cancer Ther. (2012) 11:1488-99; Herrera-Abreu et al., Early Adaptation and Acquired Resistance to CDK4/6 Inhibition in Estrogen Receptor-Positive Breast Cancer, Cancer Res. (2016) 76: 2301-2313). Cyclin E amplification also reportedly contributes to trastuzumab resistance in human epidermal growth factor receptor 2 positive (HER2+) breast cancer. (Scaltriti et al. Cyclin E amplification/overexpression is a mechanism of trastuzumab resistance in HER2+ breast cancer patients, Proc Natl Acad Sci. (2011) 108: 3761-6). Cyclin E overexpression has also been reported to play a role in basal-like and triple negative breast cancer (TNBC), as well as inflammatory breast cancer. (Elsawaf & Sinn, Triple Negative Breast Cancer: Clinical and Histological Correlations, Breast Care (2011) 6:273-278; Alexander et al., Cyclin E overexpression as a biomarker for combination treatment strategies in inflammatory breast cancer, Oncotarget (2017) 8: 14897-14911.)

Amplification or overexpression of cyclin E1 (CCNE1) is also associated with poor outcomes in ovarian, gastric, endometrial, and other cancers. (Nakayama et al., Gene amplification CCNE1 is related to poor survival and potential therapeutic target in ovarian cancer, Cancer (2010) 116: 2621-34; Etemadmoghadam et al., Resistance to CDK2 Inhibitors Is Associated with Selection of Polyploid Cells in CCNE1-Amplified Ovarian Cancer, Clin Cancer Res (2013) 19: 5960-71; Au-Yeung et al., Selective Targeting of Cyclin E1-Amplified High-Grade Serous Ovarian Cancer by Cyclin-Dependent Kinase 2 and AKT Inhibition, Clin. Cancer Res. (2017) 23:1862-1874; Ayhan et al., CCNE1 copy-number gain and overexpression identify ovarian clear cell carcinoma with a poor prognosis, Modern Pathology (2017) 30: 297-303; Ooi et al., Gene amplification of CCNE1, CCND1, and CDK6 in gastric cancers detected by multiplex ligation-dependent probe amplification and fluorescence in situ hybridization, Hum Pathol. (2017) 61: 58-67; Noske et al., Detection of CCNE1/URI (19q12) amplification by in situ hybridisation is common in high grade and type II endometrial cancer, Oncotarget (2017) 8: 14794-14805).

Mutations of CDK4 and CDK6 have been described in subgroups of melanoma and other tumors (Zuo L, et al., Germline mutations in the p16INK4a binding domain of CDK4 in familial melanoma. ;Nature Genet. (1996) 12, 97-99 Ortega S, et al. Cyclin D-dependent kinases, INK4 inhibitors and cancer. Biochim. Biophys. Acta (2002) 1602:73-87; Smalley KSM et al. Identification of a novel subgroup of melanomas with KIT/cyclin-dependent kinase-4 overexpression. Cancer Res (2008) 68: 5743-52). Amplifications of the regulatory subunits of CDKs and cyclins, and mutation, gene deletion, or transcriptional silencing of endogenous INK4 CDK inhibitors have also been reported as mechanism by which the pathway can be activated (Smalley KSM (2008)).

The development of CDK inhibitors has been reviewed in the literature. For example, see Sánchez-Martínez et al. Cyclin dependent kinase (CDK) inhibitors as anticancer drugs, Bioorg. Med. Chem. Lett. (2015) 25: 3420-3435 (and references cited therein); and Yuan et al. Selective inhibition of CDK4/6: a safe and effective strategy for developing anticancer drugs, Acta Pharmaceutica Sinica B (2020) in press, https://doi.org/10.1016/j.apsb.2020.05.001. Clinical trials for CDK4/6 inhibitors, including palbociclib, ribociclib and abemaciclib, are ongoing for breast and other cancers, as single agents or in combination with other therapeutics. Palbociclib, ribociclib and abemaciclib have been approved for treatment of hormone receptor (HR)-positive, human epidermal growth factor receptor 2 negative (HER2-) advanced or metastatic breast cancer in combination with aromatase inhibitors, such as letrozole, in a first line setting and with fulvestrant in second or later lines of therapy in certain patients. (O′Leary et al. Treating cancer with selective CDK4/6 inhibitors. Nature Reviews (2016) 13:417-430). While CDK4/6 inhibitors have shown significant clinical efficacy in ER-positive metastatic breast cancer, as with other kinases their effects may be limited over time by the development of primary or acquired resistance.

Nevertheless, there remains a need for improved therapies for the treatment of cancers. The combinations, methods and uses of the present invention are believed to have one or more advantages, such as greater efficacy than treatment with either therapeutic agent alone; potential to reduce drug-drug interactions; potential to enable an improved dosing schedule; potential to reduce side effects; potential to overcome resistance mechanisms and the like.

### BRIEF SUMMARY OF THE INVENTION

This invention relates to therapeutic methods, combinations, uses and compositions for treating abnormal cell growth, particularly cancer in a subject in need thereof.

In one aspect, the invention provides a method of treating cancer in a subject in need thereof comprising administering to the subject:
(a) an amount of a compound of Formula (I):

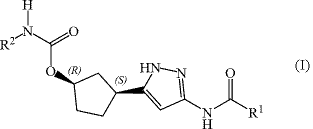

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) an amount of a cyclin dependent kinase 4/6 (CDK4/6) inhibitor;
   wherein the amounts in (a) and (b) together are effective in treating cancer.

In some embodiments of this aspect, the invention provides a method further comprising administering to the subject: (c) an amount of an additional anti-cancer agent; wherein the amounts in (a), (b) and (c) together are effective in treating cancer.

In another aspect, the invention provides a combination comprising:
(a) a compound of Formula (I):

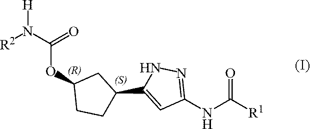

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor;
   wherein the combination of (a) and (b) is effective in treating cancer.

In some embodiments of this aspect, the combination further comprises (c) an additional anti-cancer agent; wherein the combination of (a), (b) and (c) is effective in treating cancer.

In another aspect, the invention provides a combination for use in treating cancer comprising:
(a) a compound of Formula (I):

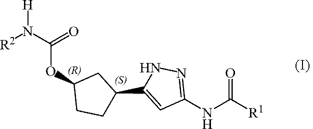

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor.

In some embodiments of this aspect, the combination for use further comprises (c) an additional anti-cancer agent.

In another aspect, the invention provides use of a combination comprising:
(a) a compound of Formula (I):

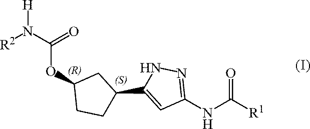

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor;
   wherein use of the combination is effective in treating cancer.

In some embodiments of this aspect, the combination further comprises (c) an additional anti-cancer agent, wherein the use of the combination of (a), (b) and (c) is effective in treating cancer.

In some embodiments of each of the combinations and uses described herein, the combination of the compound of Formula (I) and the CDK4/6 inhibitor is synergistic. In some embodiments of the combinations and uses described herein, the combination of the compound of Formula (I), the CDK4/6 inhibitor, and the additional -anti-cancer agent is synergistic.

In preferred embodiments of each of the methods, combinations and uses described herein, the CDK4/6 inhibitor is palbociclib, or a pharmaceutically acceptable salt thereof.

In some embodiments of each of the methods, combinations and uses described herein, the compound of Formula (I) is selected from the group consisting of:

(1R,3S)[3-({[3-(methoxymethyl)-1-methyl-1H-pyrazol-5-yl]carbonyl}amino)-1H-pyrazol-5-yl]cyclopentyl propan-2-ylcarbamate (COMPOUND A), having the structure:

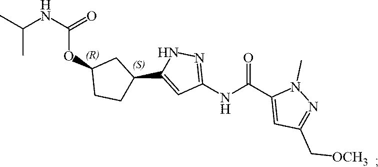

(1R,3S)[3-({[2-(methylsulfonyl)phenyl]acetyl}amino)-1H-pyrazol-5-yl]cyclopentyl (2S)-butan-2-ylcarbamate (COMPOUND B), having the structure:

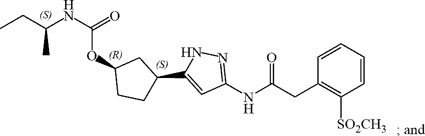

(1R,3S)(3-{[(2-methoxypyridin-4-yl)acetyl]amino}-1H-pyrazol-5-yl)cyclopentyl propylcarbamate (COMPOUND C), having the structure:

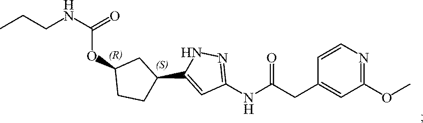

or a pharmaceutically acceptable salt thereof.

In preferred embodiments of each of the methods, combinations and uses described herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A).

In particularly preferred embodiments of each of the methods, combinations and uses described herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A), and the CDK4/6 inhibitor is palbociclib or a pharmaceutically acceptable salt thereof.

In some embodiments of each of the methods, combinations and uses described herein, the invention further comprises one or more additional anti-cancer agents. In preferred embodiments, the cancer is breast cancer (including HR+/HER2- breast cancer) and the additional anti-cancer agent is an endocrine therapeutic agent. In some such embodiments, the endocrine therapeutic agent is an aromatase inhibitor, a selective estrogen receptor degrader (SERD), or a selective estrogen receptor modulator (SERM). In preferred embodiments, the endocrine therapeutic agent is letrozole or fulvestrant.

Embodiments of each of the aspects described herein, including the methods, combinations and uses of the invention, may be combined with one or more other embodiments of the present invention described herein which is not inconsistent with the embodiment(s) with which it is combined.

### BRIEF DESCRIPTION OF THE DRAWINGS

FIG. 1. Dose matrix (A) and isobologram (B) demonstrating the effects of combining COMPOUND C and palbociclib on proliferation of HCC1428 cells.

FIG. 2. Dose matrix (A) and isobologram (B) demonstrating the effects of combining COMPOUND B and palbociclib on proliferation of HCC1428 cells.

FIG. 3. Dose matrix (A) and isobologram (B) demonstrating the effects of combining COMPOUND A and palbociclib on proliferation of HCC1428 cells.

FIG. 4. Dose matrix (A) and isobologram (B) demonstrating the effects of combining COMPOUND A and palbociclib on proliferation of MCF7 cells.

FIG. 5. Dose matrix (A) and isobologram (B) demonstrating the effects of combining COMPOUND A and palbociclib on proliferation of T47D cells.

FIG. 6. shows effects for the combination of palbociclib (10 mpk) and COMPOUND A at 25 mpk (A), 75 mpk (B) and 150 mpk (C) in the MCF-7 breast cancer xenograft model.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous).

The term “about” which used to modify a numerically defined parameter means that the parameter may vary by as much as 10% above or below the stated numerical value for that parameter. For example, a dose of “about 5 mg” means 5 mg ± 10%, i.e., the dose may vary between 4.5 mg and 5.5 mg.

The term “administration” and “treatment” as it applies to an animal, human, experimental subject, cell, tissue, organ or biological fluid, refers to contact of an exogenous pharmaceutical, therapeutic or diagnostic agent, or composition, to the animal, human, experimental subject, cell, tissue, organ or biological fluid. Treatment of a cell encompasses contact of a reagent to the cell, as well as contact of a reagent to a fluid, where the fluid is in contact with the cell. “Administration” and “treatment” also means in vitro and ex vivo treatment, e.g., of a cell, by a reagent, diagnostic, binding compound, or by another cell.

The terms “cancer”, “cancerous”, or “malignant” refer to or describe the physiological condition in mammals that is typically characterized by unregulated cell growth. As used herein “cancer” refers to any malignant and/or invasive growth or tumor caused by abnormal cell growth. As used herein “cancer” refers to solid tumors named for the type of cells that form them, as well as cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from latter one.

The term “patient” or “subject” refer to any single subject for which therapy is desired or that is participating in a clinical trial, epidemiological study or used as a control, including humans and mammalian veterinary patients such as cattle, horses, dogs, and cats. In some embodiments, the subject is a human.

In some embodiments, the subject is an adult human subject. In some embodiments, the adult subject is a woman of any menopausal status or a man. In some such embodiments, the subject is a post-menopausal woman or a man. In some such embodiments, the subject is a post-menopausal woman. In some such embodiments, the subject is a pre-menopausal or peri-menopausal woman. In some such embodiments, the subject is a pre-menopausal or peri-menopausal woman treated with a luteinizing hormone-releasing hormone (LHRH) agonist. In some such embodiments, the subject is a man. In some such embodiments, the subject is a man treated with an LHRH agonist. In some embodiments, the subject is a human child between the ages of birth and 18. In some embodiments, the subject is a child between the ages of birth and 15 having a pediatric cancer.

The terms “treat” or “treating” or “treatment” of a cancer as used herein means to administer a combination therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of a tumor; remission of the cancer; decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of patients the cancer. Positive therapeutic effects in cancer can be measured in a number of ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C <10% is considered a high anti-tumor activity level, with T/C (%) = median tumor volume of the treated / median tumor volume of the control x 100.

As used herein, the term “complete response” or “CR” means the disappearance of all signs of cancer (e.g., disappearance of all target lesions) in response to treatment. This does not always mean the cancer has been cured.

As used herein, the term “disease-free survival” (DFS) means the length of time after primary treatment for a cancer ends that the patient survives without any signs or symptoms of that cancer.

As used herein, the term “duration of response” (DoR) means the length of time that a tumor continues to respond to treatment without the cancer growing or spreading. Treatments that demonstrate improved DoR can produce a durable, meaningful delay in disease progression.

As used herein, the terms “objective response” and “overall response” refer to a measurable response, including complete response (CR) or partial response (PR). The term “overall response rate” (ORR) refers to the sum of the complete response (CR) rate and the partial response (PR) rate.

As used herein, the term “overall survival” (OS) means the length of time from either the date of diagnosis or the start of treatment for a disease, such as cancer, that patients diagnosed with the disease are still alive. OS is typically measured as the prolongation in life expectancy in patients who receive a certain treatment as compared to patients in a control group (i.e., taking either another drug or a placebo).

As used herein, the term “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

As used herein, the term “progression free survival” or “PFS” refers to the length of time during and after treatment during which the disease being treated (e.g., cancer) does not get worse. PFS, also referred to as “Time to Tumor Progression”, may include the amount of time patients have experienced a CR or PR, as well as the amount of time patients have experienced SD.

As used herein, the term “progressive disease” or “PD” refers to a cancer that is growing, spreading, or getting worse. In some embodiments, PR refers to at least a 20% increase in the SLD of target lesions, taking as reference the smallest SLD recorded since the treatment started, or to the presence of one or more new lesions.

As used herein, the term “stable disease” (SD) refers to a cancer that is neither decreasing nor increasing in extent or severity.

As used herein, the term “sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5x, 2x, 2.5x, or 3x length of the treatment duration, or longer.

The anti-cancer effect of the method of treating cancer, including “objective response,” “complete response,” “partial response,” “progressive disease,” “stable disease,” “progression free survival,” “duration of response,” as used herein, may be defined and assessed by the investigators using RECIST v1.1 (Eisenhauer et al., New response evaluation criteria in solid tumours: Revised RECIST guideline (version 1.1), Eur J of Cancer, 2009; 45(2):228-47).

of the therapeutic effect of the methods, combinations and uses herein, may be defined by reference to any of the following: complete response (CR), disease free survival (DFS), duration of response (DoR), overall response rate (ORR), overall survival (OS), partial response (PR), or progression free survival (PFS). In some embodiments, response to a combination of the invention is any of PR, CR, PFS, DFS, OR or OS that is assessed using Response Evaluation Criteria in Solid Tumors (RECIST) 1.1 response criteria.

In some embodiments, the methods, combinations and uses herein relate to neoadjuvant therapy, adjuvant therapy, first-line therapy, second-line therapy, or third-line or later lines of therapy. In each case as further described herein, the cancer may be localized, advanced or metastatic, and the intervention may occur at point along the disease continuum (i.e., at any stage of the cancer).

The treatment regimen for a method, combination or use of the invention that is effective to treat cancer in a subject may vary according to factors such as the disease state, age, and weight of the subject, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as the Student’s t-test, the chi2-test the U-test according to Mann and Whitney, the Kruskal-Wallis test (H-test), Jonckheere-Terpstrat-test and the Wilcon on-test.

The terms “treatment regimen”, “dosing protocol” and “dosing regimen” may be used interchangeably to refer to the dose and timing of administration of each therapeutic agent in a combination of the invention.

“Ameliorating” means a lessening or improvement of one or more symptoms upon treatment with a combination described herein, as compared to not administering the combination. “Ameliorating” also includes shortening or reduction in duration of a symptom.

As used herein, an “effective dosage” or “effective amount” of a compound or pharmaceutical composition is an amount sufficient to affect any one or more beneficial or desired outcomes, including biochemical, histological and / or behavioral symptoms, of the disease, its complications and intermediate pathological phenotypes presenting during development of the disease.

For therapeutic use, a “therapeutically effective amount” refers to that amount of a compound or combination being administered which will relieve to some extent one or more of the symptoms of the disorder being treated. In reference to the treatment of cancer, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) relieving to some extent (or, preferably, eliminating) one or more signs or symptoms associated with the cancer, (5) decreasing the dose of other medications required to treat the disease, and/or (6) enhancing the effect of another medication, and/or (7) delaying the progression of the disease in a patient.

An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound or pharmaceutical composition may or may not be achieved in conjunction with another drug, compound, or pharmaceutical composition.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukaemia’s (cancers of the blood) generally do not form solid tumors (National Cancer Institute, Dictionary of Cancer Terms).

“Tumor burden” or “tumor load’, refers to the total amount of tumorous material distributed throughout the body. Tumor burden refers to the total number of cancer cells or the total size of tumor(s), throughout the body, including lymph nodes and bone marrow. Tumor burden can be determined by a variety of methods known in the art, such as, e.g., using callipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using callipers, or while in the body using imaging techniques, e.g., bone scan, ultrasound, CR or MRI scans.

The term “additive” is used to mean that the result of the combination of two compounds, components or targeted agents is no greater than the sum of each compound, component, or targeted agent individually.

The term “synergy” or “synergistic” are used to mean that the result of the combination of two or more compounds, components or targeted agents is greater than the sum of each compound, component, or targeted agent individually. This improvement in the disease, condition or disorder being treated is a “synergistic” effect and combinations providing a synergistic effect may be referred to as synergistic combinations. A “synergistic amount” is an amount of the combination of the two compounds, components or targeted agents that results in a synergistic effect, as “synergistic” is defined herein. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol. Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively. Ma & Motsinger-Reif, Current Method for Quantifying Drug Synergism, Proteom. Bioinform (2019) 1(2):43-48; Tang et al., What is Synergy? The Saariselkä Agreement Revisited, Front Pharmacol. 2015) Article 181, 6: 1-5.

A number of CDK4/6 inhibitors have been approved or are currently in clinical development, including: palbociclib (also known as PD-0332991 or PF-00080665), ribociclib (also known as LEE-011), abemaciclib (also known as LY2835219), lerociclib (also known as G1T38) and trilaciclib (also known as GTI128). Such compounds, or their pharmaceutically acceptable salts, may be useful as CDK4/6 inhibitors in the present invention. CDK4/6 inhibitors may be identified using standard assays routinely used to measure inhibition of CDKs and other protein kinases. See, e.g., Fry et al., Cell cycle and biochemical effects of PD 0183812. A potent inhibitor of the cyclin D-dependent kinases CDK4 and CDK6, J. Biol. Chem. (2001), 276: 16617-16623.

In some embodiments of each of the methods, combinations and uses described herein, the CDK4/6 inhibitor is selected from the group consisting of palbociclib, ribociclib, abemaciclib, lerociclib and trilaciclib, or a pharmaceutically acceptable salt thereof. In some embodiments of each of the methods, combinations and uses described herein, the CDK4/6 inhibitor is selected from the group consisting of palbociclib, ribociclib and abemaciclib, or a pharmaceutically acceptable salt thereof. In preferred embodiments of each of the methods, combinations and uses described herein, the CDK4/6 inhibitor is palbociclib, or a pharmaceutically acceptable salt thereof. In other embodiments of each of the methods, combinations and uses described herein, the CDK4/6 inhibitor is ribociclib, or a pharmaceutically acceptable salt thereof. In other embodiments of each of the methods, combinations and uses described herein, the CDK4/6 inhibitor is abemaciclib, or a pharmaceutically acceptable salt thereof. In still other embodiments of each of the methods, combinations and uses described herein, the CDK4/6 inhibitor is lerociclib or trilaciclib, or a pharmaceutically acceptable salt thereof.

Palbociclib, or 6-acetyl-8-cyclopentyl-5-methyl-2-(5-piperazin-1-yl-pyridin-2-ylamino)-8H-pyrido[2,3-d]pyrimidin-7-one (also referred to as PD-0332991) is a potent and selective inhibitor of CDK4 and CDK6, having the structure:

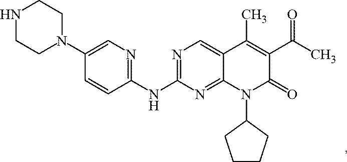

Palbociclib is described in WHO Drug Information, Vol. 27, No. 2, page 172 (2013). Palbociclib and pharmaceutically acceptable salts and formulations thereof are disclosed in International Publication No. WO 2003/062236 and U.S. Pat. Nos. 6,936,612, 7,456,168 and RE47,739; International Publication No. WO 2005/005426 and U.S. Pat. Nos. 7,345,171 and 7,863,278; International Publication No. WO 2008/032157 and U.S. Pat. No. 7,781,583; International Publication No. WO 2014/128588 and U.S. Pat. Publication No. 2018/0065964; and International Publication No. WO 2016/193860 and U.S. Pat. Publication No. 2018/0207100. The contents of each of the foregoing references are incorporated herein by reference in their entirety.

Ribociclib is described in WHO Drug Information, Vol. 29, No. 1, pages 108-109 (2015). Abemaciclib is described in WHO Drug Information, Vol. 29, No. 3, page 386 (2015). Trilaciclib is described in WHO Drug Information, Vol. 32, No. 1, pages 176-177 (2018). Lerociclib is described in WHO Drug Information, Vol. 32, No. 4, page 608 (2018).

All references herein to CDK2 inhibitors of Formula (I), or to CDK4/6 inhibitors include (to the extent chemically feasible) references to pharmaceutically acceptable salts, solvates, hydrates and complexes thereof, and to solvates, hydrates and complexes of pharmaceutically acceptable salts thereof, and include amorphous and polymorphic forms, stereoisomers, and isotopically labeled versions thereof.

As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein. For example, the compounds of the invention that are basic in nature may be capable of forming a wide variety of salts with various inorganic and organic acids. The acids that may be used to prepare pharmaceutically acceptable acid addition salts of such basic compounds of those that form non-toxic acid addition salts, i.e., salts containing pharmacologically acceptable anions. Examples of anions suitable for mono- and di- acid addition salts include, but are not limited to, acetate, asparatate, benzenesulfonate, benzoate, besylate, bicarbonate, bisulfate, bitartrate, bromide, calcium edetate, camsylate, carbonate, chloride, citrate, decanoate, edetate, edislyate, estolate, esylate, fumarate, gluceptate, gluconate, glutamate, glycollate, hexanoate, hexylresorcinate, hydrabamine, hydroxynaphthoate, iodide, isethionate, lactate, lactobionate, malate, maleate, mandelate, mesylate, methylsulfate, mucate, napsylate, nitrate, octanoate, oleate, pamoate (embonate), pantothenate, phosphate, polygalacturonate, propionate, salicylate, stearate, subacetate, succinate, sulfate, tannate, tartrate, teoclate, tosylate, triethiodode, and valerate salts. Alternatively, compounds that are acidic in nature may be capable of forming base salts with various pharmacologically acceptable cations which form non-toxic base salts. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. Examples of cations suitable for such salts include alkali metal or alkaline-earth metal salts and other cations, including aluminium, arginine, benzathine, calcium, chloroprocaine, choline, diethanolamine, ethanolamine, ethylenediamine, lysine, magnesium, histidine, lithium, meglumine, potassium, procaine, sodium, triethyamine and zinc. Salts may be prepared by conventional techniques. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002). Methods for making pharmaceutically acceptable salts are known to those of skill in the art.

### Therapeutic Methods, Combinations, Uses

The present invention provides methods, combinations and uses that may be useful for treating cancer. Some embodiments provided herein result in one or more of the following effects: (1) inhibiting cancer cell proliferation; (2) inhibiting cancer cell invasiveness; (3) inducing apoptosis of cancer cells; (4) inhibiting cancer cell metastasis; (5) inhibiting angiogenesis; or (6) overcoming one or more resistance mechanisms relating to a cancer treatment.

The present invention provides methods, combinations and uses comprising a compound of Formula (I):

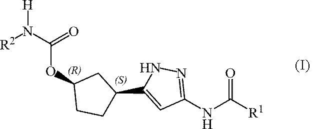

or a pharmaceutically acceptable salt thereof, wherein:
R¹ is —L—(5-6 membered heteroaryl) or —L—(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy.

In each instance recited herein, reference to “a compound of Formula (I)” may be replaced by “a CDK2 inhibitor of Formula (I).”

In one aspect, the invention provides a method of treating cancer in a subject in need thereof comprising administering to the subject:
(a) an amount of a compound of Formula (I):

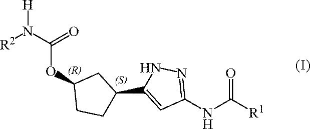

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where
   each C₁-C₄ alkyl is optionally substituted by F, OH or C₁-C₄ alkoxy; and
(b) an amount of a cyclin dependent kinase 4/6 (CDK4/6) inhibitor;
   wherein the amounts in (a) and (b) together are effective in treating cancer.

In some embodiments of this aspect, the invention provides a method further comprising administering to the subject (c) an amount of an additional anti-cancer agent; wherein the amounts in (a), (b) and (c) together are effective in treating cancer.

In another aspect, the invention provides a combination comprising:
(a) a compound of Formula (I):

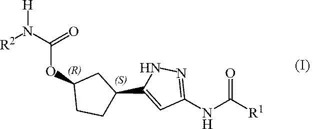

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor;
   wherein the combination of (a) and (b) is effective in treating cancer.

In some embodiments of this aspect, the combination further comprises (c) an additional anti-cancer agent; wherein the combination of (a), (b) and (c) is effective in treating cancer.

In another aspect, the invention provides a combination for use in treating cancer comprising:
(a) a compound of Formula (I):

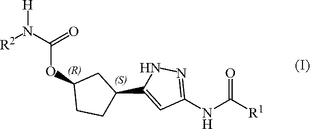

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor.

In some embodiments of this aspect, the combination for use further comprises (c) an additional anti-cancer agent.

In another aspect, the invention provides use of a combination comprising:
(a) a compound of Formula (I):

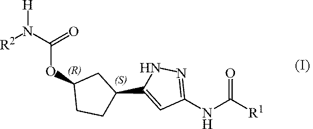

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor;
   wherein use of the combination of (a) and (b) is effective in treating cancer.

In some embodiments of this aspect, the combination further comprises (c) an additional anti-cancer agent, wherein the use of the combination of (a), (b) and (c) is effective in treating cancer.

In some embodiments of each of the methods, combinations and uses described herein, the compound of Formula (I) is selected from the group consisting of:

(1R,3S)[3-({[3-(methoxymethyl)-1-methyl-1H-pyrazol-5-yl]carbonyl}amino)-1H-pyrazol-5-yl]cyclopentyl propan-2-ylcarbamate (COMPOUND A), having the structure:

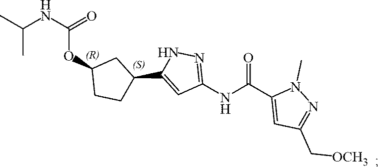

(1R,3S)[3-({[2-(methylsulfonyl)phenyl]acetyl}amino)-1H-pyrazol-5-yl]cyclopentyl (2S)-butan-2-ylcarbamate (COMPOUND B), having the structure:

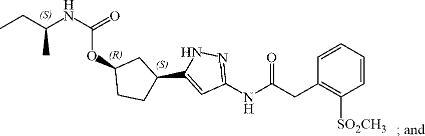

(1R,3S)(3-{[(2-methoxypyridin-4-yl)acetyl]amino}-1H-pyrazol-5-yl)cyclopentyl propylcarbamate (COMPOUND C), having the structure:

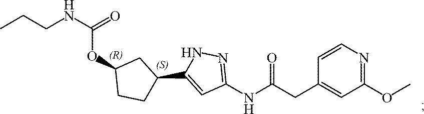

or a pharmaceutically acceptable salt thereof.

In preferred embodiments of each of the methods, combinations and uses described herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) having the structure:

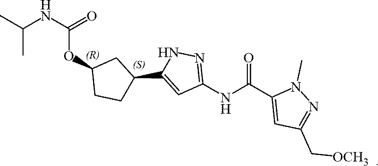

In preferred embodiments of each of the methods, combinations and uses described herein, the CDK4/6 inhibitor is palbociclib or a pharmaceutically acceptable salt thereof. In some such embodiments, the CDK4/6 inhibitor is palbociclib. In some embodiments of each of the methods, combinations and uses described herein, the CDK4/6 inhibitor is selected from the group consisting of palbociclib, ribociclib and abemaciclib, or a pharmaceutically acceptable salt thereof.

In particularly preferred embodiments of each of the methods, combinations and uses described herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A), and the CDK4/6 inhibitor is palbociclib or a pharmaceutically acceptable salt thereof.

In another aspect, the invention provides a method of treating cancer in a subject in need thereof comprising administering to the subject:
(a) an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A); and
(b) an amount of a cyclin dependent kinase 4/6 (CDK4/6) inhibitor;
   wherein the amounts in (a) and (b) together are effective in treating cancer.

In another aspect, the invention provides a method of treating cancer in a subject in need thereof comprising administering to the subject:
(a) an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A);
(b) an amount of a cyclin dependent kinase 4/6 (CDK4/6) inhibitor; and
(c) an amount of an additional anti-cancer agent;
   wherein the amounts in (a), (b) and (c) together are effective in treating cancer.

In another aspect, the invention provides a method of treating cancer in a subject in need thereof comprising administering to the subject:
(a) an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A); and
(b) an amount of palbociclib or a pharmaceutically acceptable salt thereof;
   wherein the amounts in (a) and (b) together are effective in treating cancer.

In another aspect, the invention provides a method of treating cancer in a subject in need thereof comprising administering to the subject:
(a) an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A);
(b) an amount of palbociclib or a pharmaceutically acceptable salt thereof; and
(c) an amount of an additional anti-cancer agent;
   wherein the amounts in (a), (b) and (c) together are effective in treating cancer.

In another aspect, the invention provides a combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A); and
(b) a cyclin dependent kinase 4/6 (CDK4/6) inhibitor;
   wherein the combination of (a) and (b) is effective in treating cancer.

In another aspect, the invention provides a combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A);
(b) a cyclin dependent kinase 4/6 (CDK4/6) inhibitor; and
(c) an additional anti-cancer agent;
   wherein the combination of (a), (b) and (c) is effective in treating cancer.

In another aspect, the invention provides a combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A); and
(b) palbociclib or a pharmaceutically acceptable salt thereof;
   wherein the combination of (a) and (b) is effective in treating cancer.

In another aspect, the invention provides a combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A);
(b) palbociclib or a pharmaceutically acceptable salt thereof; and
(c) an additional anti-cancer agent;
   wherein the combination of (a), (b) and (c) is effective in treating cancer.

In another aspect, the invention provides a combination for use in treating cancer comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A); and
(b) a cyclin dependent kinase 4/6 (CDK4/6) inhibitor.

In another aspect, the invention provides a combination for use in treating cancer comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A);
(b) a cyclin dependent kinase 4/6 (CDK4/6) inhibitor; and
(c) an additional anti-cancer agent.

In another aspect, the invention provides a combination for use in treating cancer comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A); and
(b) palbociclib or a pharmaceutically acceptable salt thereof.

In another aspect, the invention provides a combination for use in treating cancer comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A);
(b) palbociclib or a pharmaceutically acceptable salt thereof; and
(c) an additional anti-cancer agent.

In another aspect, the invention provides use of a combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A); and
(b) a cyclin dependent kinase 4/6 (CDK4/6) inhibitor;
   wherein use of the combination of (a) and (b) is effective in treating cancer.

In another aspect, the invention provides use of a combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A);
(b) a cyclin dependent kinase 4/6 (CDK4/6) inhibitor; and
(c) an additional anti-cancer agent;
   wherein use of the combination of (a), (b) and (c) is effective in treating cancer.

In another aspect, the invention provides use of a combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A); and
(b) palbociclib or a pharmaceutically acceptable salt thereof;
   wherein use of the combination of (a) and (b) is effective in treating cancer.

In another aspect, the invention provides use of a combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A);
(b) palbociclib or a pharmaceutically acceptable salt thereof; and
(c) an additional anti-cancer agent.
   wherein use of the combination of (a), (b) and (c) is effective in treating cancer.

In some embodiments of each of the methods, combinations and uses described herein, the cancer is selected from the group consisting of breast cancer, lung cancer, ovarian cancer, peritoneal cancer, fallopian tube cancer, bladder cancer, uterine cancer, colon cancer, prostate cancer, esophageal cancer, liver cancer, pancreatic cancer and stomach cancer.

In some embodiments of each of the methods, combinations and uses described herein, the cancer is characterized by amplification or overexpression of cyclin E1 (CCNE1) and/or cyclin E2 (CCNE2). In some such embodiments, the cancer is characterized by amplification or overexpression of cyclin E1 (CCNE1).

Examples of cyclin E-dominant cancers include, but are not limited to, ovarian cancer, breast cancer, liver cancer, stomach cancer, esophageal cancer, bladder cancer, uterine cancer, small cell lung cancer (SCLC), non-small cell lung cancer (NSCLC), colon cancer, prostate cancer or pancreatic cancer.

The retinoblastoma gene product, RB, may be mutated or deleted in several tumor types, such as retinoblastoma, osteosarcoma and small-cell lung cancer (SCLC), prostate cancer, uterine cancer, bladder cancer, liver cancer, ovarian cancer, esophogeal cancer, stomach cancer, cervical cancer, glioblastoma, non-small cell lung cancer (NSCLC), lymphoma, breast cancer, and head and neck cancer. In human cancers, the function of RB may be disrupted through neutralization by a binding protein, (e.g., the human papilloma virus-E7 protein in cervical carcinoma; Ishiji, T, 2000, J Dermatol., 27: 73-86) or deregulation of pathways ultimately responsible for its phosphorylation.

By “RB pathway” it is meant the entire pathway of molecular signaling that includes retinoblastoma protein (RB), and other protein/protein families in the pathway, including but not limited to CDK, E2f, atypical protein kinase C, and Skp2. Inactivation of the RB pathway often results from perturbation of p16INK4a, Cyclin D1, and CDK4.

The terms “RB+,” “RB plus” or “RB-positive” may be used to describe cells expressing detectable amounts of functional RB protein. RB positive includes wild-type and non-mutated RB protein. A wild-type RB (RB-WT) is generally understood to mean that form of the RB protein which is normally present in a corresponding population and which has the function which is currently assigned to this protein. RB positive may be cells which contain a functional RB gene. Cells which are RB positive may also be cells that can encode a detectable RB protein function.

The terms “RB-,” “RB minus,” “RB deficient” or “RB-negative” describe several types of cell where the function of RB is disrupted, including cells which produce no detectable amounts of functional RB protein. Cells that are RB negative may be cells which do not contain a functional RB gene. Cells that are RB negative may also be cells that can encode an RB protein, but in which the protein does not function properly.

In some embodiments of each of the methods, combinations and uses described herein, the cancer is characterized as retinoblastoma wild type (RB WT). In some embodiments of each of the methods, combinations and uses described herein, the cancer is characterized as RB-positive. RB-positive tumors contain at least some functional retinoblastoma genes.

In some embodiments of each of the methods, combinations and uses described herein, the cancer is characterized as RB-negative. RB-negative cancers may be characterized by loss of function mutations, which may encodemissense mutations (i.e., encode the wrong amino acid) or nonsense mutatons (i.e., encode a stop codon). Alternatively, RB-negative cancers may be characterized by deletion of all or part of the retinoblastoma gene.

In some embodiments of each of the methods, combinations and uses described herein, the cancer is advanced or metastatic cancer.

In some embodiments of each of the methods, combinations, and uses described herein, the cancer is refractory, i.e., the cancer does not respond at all to treatment with a therapeutic agent or class (including a standard of care agent or class) or initially responds but starts to grow again in a very short period of time.

In some embodiments of each of the methods, combinations, uses described herein, the cancer is resistant to a therapeutic agent or class (including a standard of care agent or class). In some embodiments of each of the methods, combinations, uses described herein, the cancer is characterized by innate or acquired resistance to a therapeutic agent or class (including a standard of care agent or class).

In some embodiments of each of the methods, combinations and uses described herein, the compound of Formula (I) or a pharmaceutically acceptable salt thereof, and the CDK4/6 inhibitor or a pharmaceutically acceptable salt thereof, are administered sequentially, simultaneously, or concurrently. In some embodiments of each of the methods, combinations and uses described herein, COMPOUND A, and the CDK4/6 inhibitor or a pharmaceutically acceptable salt thereof, are administered sequentially, simultaneously, or concurrently. In some embodiments of each of the methods, combinations and uses described herein, COMPOUND A, and palbociclib or a pharmaceutically acceptable salt thereof, are administered sequentially, simultaneously, or concurrently.

In preferred embodiments of the methods, combinations and uses described herein, the cancer is breast cancer as further described herein. In some embodiments of each of the breast cancer subtypes described herein, the breast cancer is advanced or metastatic breast cancer.

In some embodiments, the breast cancer is hormone receptor positive (HR+), i.e., the breast cancer is estrogen receptor positive (ER+) and/or progesterone receptor positive (PR+). In some embodiments, the breast cancer is hormone receptor negative (HR-), i.e., the breast cancer is estrogen receptor negative (ER-) and progesterone receptor negative (PR-).

In some embodiments wherein the cancer is HR+ breast cancer, the methods, combinations and uses described herein further comprise an additional anti-cancer agent, wherein the additional anti-cancer agent is an endocrine therapeutic agent. In some such embodiments, the endocrine therapeutic agent is an aromatase inhibitor, a SERD or a SERM. In preferred embodiments, the endocrine therapeutic agent is letrozole or fulvestrant.

In some embodiments, the breast cancer is human epidermal growth factor receptor 2 negative (HER2-). In some embodiments, the breast cancer is human epidermal growth factor receptor 2 positive (HER2+).

In some embodiments, the breast cancer is associated with the BRCA1 or BRCA2 gene.

In preferred embodiments, the breast cancer is HR+/HER2- breast cancer. In some such embodiments, the HR+/HER2- breast cancer is refractory to treatment with a CDK4/6 inhibitor, such as palbociclib or a pharmaceutically acceptable salt thereof. In some such embodiments, the HR+/HER2- breast cancer is resistant to treatment with a CDK4/6 inhibitor, such as palbociclib or a pharmaceutically acceptable salt thereof. In some embodiments, the HR+/HER2- breast cancer is characterized by amplification or overexpression of cyclin E1 (CCNE1) and/or cyclin E2 (CCNE2). In some embodiments, the HR+/HER2- breast cancer is characterized by amplification or overexpression of cyclin E1 (CCNE1). In some embodiments of each of the foregoing, the HR+/HER2-breast cancer is advanced or metastatic HR+/HER2- breast cancer.

In some embodiments, the breast cancer is HR+/HER2+ breast cancer. In other embodiments, the breast cancer is HR-/HER2+ breast cancer. In some embodiments wherein the breast cancer is HER2+, the methods, combinations and uses described herein further comprise an additional anti-cancer agent, wherein the additional anti-cancer agent is a HER2-targeted agent (e.g., trastuzumab emtansine, fam-trastuzumab deruxtecan, pertuzumab, lapatinib, neratinib or tucatinib), or an agent targeting the PI3K/AKT molecular pathway (e.g., ipatasertib).

In other embodiments, the breast cancer is triple negative breast cancer (TNBC), i.e., the breast cancer is ER-, PR- and HER2-. In some embodiments, the TNBC is refractory to treatment with a CDK4/6 inhibitor, such as palbociclib or a pharmaceutically acceptable salt thereof. In some embodiments, the TNBC is resistant to treatment with a CDK4/6 inhibitor, such as palbociclib or a pharmaceutically acceptable salt thereof. In some embodiments, the TNBC is characterized by amplification or overexpression of cyclin E1 (CCNE1) and/or cyclin E2 (CCNE2). In some such embodiments, the TNBC is characterized by amplification or overexpression of cyclin E1 (CCNE1). In some embodiments, the TNBC is locally recurrent/advanced or metastatic TNBC. In some embodiments, the TNBC is advanced or metastatic TNBC.

In some embodiments of each of the foregoing, the breast cancer is refractory to treatment with one or more standard of care agents. In some embodiments of each of the foregoing, the breast cancer is resistant to treatment with one or more standard of care agents.

In some such embodiments, the breast cancer is refractory or resistant to treatment with endocrine therapeutic agents, such as aromatase inhibitors, SERDs, or SERMs. In some embodiments, the breast cancer is refractory or resistant to treatment with a CDK4/6 inhibitor. For example, in some embodiments, the breast cancer is refractory or resistant to treatment with palbociclib or a pharmaceutically acceptable salt thereof. In other embodiments, the breast cancer is refractory or resistant to treatment with, or has progressed on, treatment with antineoplastic chemotherapeutic agents such as platinum agents, taxanes, anthracyclines or anti-metabolites.

In some embodiments of each of the methods, combinations and uses described herein, the cancer is lung cancer. In some such embodiments, the lung cancer is advanced or metastatic lung cancer.

In some such embodiments, the lung cancer is small cell lung cancer (SCLC). In some such embodiments, the SCLC is characterized by loss of retinoblastoma (RB) function. In some such embodiments, the SCLC is advanced or metastatic SCLC. In some such embodiments, the SCLC is advanced or metastatic SCLC characterized by loss of retinoblastoma (RB) function.

In other such embodiments, the lung cancer is non-small cell lung cancer (NSCLC). In some such embodiments, the NSCLC is characterized by amplification or overexpression of cyclin E1 (CCNE1) and/or cyclin E2 (CCNE2). In some embodiments, the NSCLC is characterized by amplification or overexpression of cyclin E1 (CCNE1). In some such embodiments, the NSCLC is advanced or metastatic NSCLC. In some such embodiments, the NSCLC is advanced or metastatic NSCLC characterized by amplification or overexpression of cyclin E1 (CCNE1).

In some embodiments, the cancer is lung cancer, including SCLC or NSCLC, and the methods, combinations and uses described herein further comprise an additional anti-cancer agent.

In some embodiments of each of the methods, combinations and uses described herein, the cancer is ovarian cancer, peritoneal cancer, or fallopian tube cancer (FTC). In some such embodiments, the ovarian cancer is epithelial ovarian cancer (EOC). In some such embodiments, the peritoneal cancer is primary peritoneal carcinomatosis (PPC). In some embodiments, the cancer is epithelial ovarian cancer (EOC), primary peritoneal carcinomatosis (PPC) or fallopian tube cancer (FTC). In some embodiments, the ovarian cancer is persistent, refractory or recurrent ovarian cancer. In some embodiments, the ovarian cancer is platinum resistant ovarian cancer. In some such embodiments, the cancer is advanced or metastatic ovarian cancer. In some such embodiments, the cancer is platinum resistant advanced or metastatic ovarian cancer. In some such embodiments, the cancer is advanced or metastatic EOC, PPC or FTC. In some such embodiments, the cancer is platinum resistant advanced or metastatic EOC, PPC or FTC.

In some embodiments, each of the methods, combinations and uses described herein further comprise an additional anti-cancer agent. In some embodiments the additional anti-cancer agent is a standard of care agent for the type of cancer. In some embodiments, each of the methods, combinations and uses described herein further comprise an additional anti-cancer agent, wherein the compound of Formula (I) or a pharmaceutically acceptable salt thereof, the CDK4/6 inhibitor or a pharmaceutically acceptable salt thereof, and the additional anti-cancer agent are administered sequentially, simultaneously or concurrently.

In some embodiments of each of the methods, combinations and uses described herein, the cancer is breast cancer (including HR+ or HR+/HER2- breast cancer), and the methods, combinations and uses further comprise an additional anti-cancer agent. In some embodiments, the additional anti-cancer agent is an endocrine therapeutic agent. In some such embodiments, the endocrine therapeutic agent is an aromatase inhibitor, a SERD, or a SERM. In some embodiments, the endocrine therapeutic agent is an aromatase inhibitor. In some such embodiments, the aromatase inhibitor is selected from the group consisting of letrozole, anastrozole, and exemestane. In some preferred embodiments, the aromatase inhibitor is letrozole. In some embodiments, the endocrine therapeutic agent is a SERD. In some such embodiments, the SERD is selected from the group consisting of fulvestrant, elacestrant (RAD-1901, Radius Health), SAR439859 (Sanofi), RG6171 (Roche), AZD9833 (AstraZeneca), AZD9496 (AstraZeneca), rintodestrant (G1 Therapeutics), ZN-c5 (Zentalis), LSZ102 (Novartis), D-0502 (Inventisbio), LY3484356 (Lilly), and SHR9549 (Jiansu Hengrui Medicine). In some preferred embodiments, the SERD is fulvestrant. In some embodiments, the endocrine therapeutic agent is a SERM. In some such embodiments, the SERM is selected from the group consisting of tamoxifen, raloxifene, toremifene, lasofoxifene, bazedoxifene and afimoxifene. In some such embodiments, the SERM is tamoxifen or raloxifene.

In some embodiments of each of the methods, combinations and uses described herein, the cancer is breast cancer (including HR+ or HR+/HER2- breast cancer), and the methods, combinations and uses further comprise an additional anti-cancer agent, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A), the CDK4/6 inhibitor is palbociclib or a pharmaceutically acceptable salt thereof, and the additional anti-cancer agent is an endocrine therapeutic agent, wherein the endocrine therapeutic agent is an aromatase inhibitor, a SERD, or a SERM. In some such embodiments, the endocrine therapeutic agent is letrozole or fulvestrant.

### Pharmaceutical Compositions, Medicaments and Kits

The present invention further provides pharmaceutical compositions, medicaments and kits comprising a compound of Formula (I):

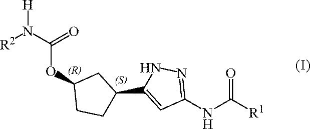

or a pharmaceutically acceptable salt thereof, wherein:
R¹ is —L—(5-6 membered heteroaryl) or —L—(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy.

In another aspect, the invention provides a pharmaceutical composition comprising a compound of Formula (I) or a pharmaceutically acceptable salt thereof, a CDK4/6 inhibitor or a pharmaceutically acceptable salt thereof, and a pharmaceutically acceptable carrier or excipient. In some embodiments of this aspect, the pharmaceutical composition further comprises an additional anti-cancer agent (e.g., an endocrine therapeutic agent).

In another aspect, the invention provides a first pharmaceutical composition comprising a compound of Formula (I) or a pharmaceutically acceptable salt thereof, and a pharmaceutically acceptable carrier or excipient, and a second pharmaceutical composition comprising a CDK4/6 inhibitor or a pharmaceutically acceptable salt thereof, and a pharmaceutically acceptable carrier or excipient, wherein the first and second pharmaceutical compositions are administered sequentially, simultaneously or concurrently. Some embodiments of this aspect further comprise a third pharmaceutical composition comprising an additional anti-cancer agent (e.g., an endocrine therapeutic agent) and a pharmaceutically acceptable carrier or excipient, wherein the first, second and third pharmaceutical compositions are administered sequentially, simultaneously, or concurrently.

In another aspect, the invention provides a combination comprising a compound of Formula (I) or a pharmaceutically acceptable salt thereof, and a CDK4/6 inhibitor or a pharmaceutically acceptable salt thereof, for use in the manufacture of a medicament for treating cancer in a subject. In another aspect, the invention provides use of a combination comprising a compound of Formula (I) or a pharmaceutically acceptable salt thereof, and a CDK4/6 inhibitor or a pharmaceutically acceptable salt thereof, in the manufacture of a medicament for treating cancer in a subject. In some embodiments of these aspects, the combination further comprises an additional anti-cancer agent (e.g., an endocrine therapeutic agent) for use in the manufacture of a medicament.

In another aspect, the invention provides a compound of Formula (I) or a pharmaceutically acceptable salt thereof for use in the manufacture of a medicament for treating cancer, wherein the medicament is adapted for use in combination with a CDK4/6 inhibitor or a pharmaceutically acceptable salt thereof. In another aspect, the invention provides a compound of Formula (I) or a pharmaceutically acceptable salt thereof for use in the manufacture of a medicament for treating cancer, wherein the medicament is adapted for use in combination with a CDK4/6 inhibitor or a pharmaceutically acceptable salt thereof, and an additional anti-cancer agent, e.g., an endocrine therapeutic agent. In another aspect, the invention provides use of a compound of Formula (I) or a pharmaceutically acceptable salt thereof, in the manufacture of a medicament for treating cancer, wherein the medicament is adapted for use in combination with a CDK4/6 inhibitor or a pharmaceutically acceptable salt thereof. In another aspect, the invention provides use of a compound of Formula (I) or a pharmaceutically acceptable salt thereof, in the manufacture of a medicament for treating cancer, wherein the medicament is adapted for use in combination with a CDK4/6 inhibitor or a pharmaceutically acceptable salt thereof, and an additional anti-cancer agent (e.g., an endocrine therapeutic agent).

In preferred embodiments of each of the pharmaceutical compositions and medicaments described herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A).

In preferred embodiments of each of the pharmaceutical compositions and medicaments described herein, the CDK4/6 inhibitor is palbociclib or a pharmaceutically acceptable salt thereof.

In particularly preferred embodiments of each of pharmaceutical compositions and medicaments described herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A), and the CDK4/6 inhibitor is palbociclib or a pharmaceutically acceptable salt thereof.

In another aspect, the invention provides a kit comprising a first container, a second container and a package insert, wherein the first container comprises at least one dose of a compound of Formula (I) or a pharmaceutically acceptable salt thereof, as further described herein; the second container comprises at least one dose of a CDK4/6 inhibitor or a pharmaceutically acceptable salt thereof; and the package insert comprises instructions for treating cancer in a subject using the medicaments. In another aspect, the invention provides a kit comprising a first container, a second container, a third container, and a package insert, wherein the first container comprises at least one dose of a compound of Formula (I) or a pharmaceutically acceptable salt thereof; the second container comprises at least one dose of a CDK4/6 inhibitor or a pharmaceutically acceptable salt thereof; the third container comprises at least one dose of an additional anti-cancer agent (e.g., an endocrine therapeutic agent); and the package insert comprises instructions for treating cancer in a subject using the medicaments.

In preferred embodiments of the kits herein, the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A). In preferred embodiments of the kits herein, the CDK4/6 inhibitor is palbociclib or a pharmaceutically acceptable salt thereof.

In particularly preferred embodiments of the kits herein, the compound of Formula (I) is (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A), and the CDK4/6 inhibitor is palbociclib or a pharmaceutically acceptable salt thereof.

In embodiments of the pharmaceutical compositions, medicaments, and kits comprising an additional anti-cancer agent, the additional anti-cancer agent is an endocrine therapeutic agent, such as an aromatase inhibitor, a SERD, or a SERM. In some such embodiments, the endocrine therapeutic agent is letrozole or fulvestrant.

The pharmaceutical compositions, medicaments and kits described herein may be useful for treating the cancers described above with respect to the methods, combinations and uses of the invention. In some embodiments, the pharmaceutical compositions, medicaments and kits may be useful for treating cancer selected from the group consisting of breast cancer (including HR+/HER2-, HR+/HER2+, HR-/HER2+ or TNBC), lung cancer (including SCLC or NSCLC), ovarian cancer (including EOC), peritoneal cancer (including PPC), fallopian tube cancer (including FTC), bladder cancer, colon cancer, uterine cancer, prostate cancer, esophageal cancer, liver cancer, pancreatic cancer and stomach cancer.

### Additional Methods and Uses

The invention further provides methods and uses comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) as a single agent, optionally in combination with an additional anti-cancer agent, as further described below.

In one aspect, the invention provides a method of treating cancer in a subject in need thereof comprising administering to the subject a therapeutically effective amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A). In some embodiments the invention provides a method of treating cancer comprising administering to the subject a therapeutically effective amount of COMPOUND A, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

In another aspect, the invention provides a method of treating lung cancer in a subject in need thereof comprising administering to the subject a therapeutically effective amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A). In some embodiments of this aspect, the lung cancer is small cell lung cancer (SCLC). In some such embodiments the SCLC is RB-negative. In some embodiments of this aspect, the lung cancer is non-small cell lung cancer (NSCLC). In some embodiments of this aspect, the lung cancer is advanced or metastatic lung cancer. In some such embodiments, the lung cancer is advanced or metastatic SCLC. In some such embodiments, the lung cancer is advanced or metastatic NSCLC.

In another aspect, the invention provides a method of treating ovarian cancer, peritoneal cancer, or fallopian tube cancer in a subject in need thereof comprising administering to the subject a therapeutically effective amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A). In some embodiments, the cancer is ovarian cancer. In some such embodiments, the ovarian cancer is epithelial ovarian cancer (EOC). In some such embodiments, the ovarian cancer is advanced or metastatic ovarian cancer (including EOC). In some such embodiments, the ovarian cancer is platinum resistant ovarian cancer (including EOC). In some such embodiments, the ovarian cancer is platinum resistant advanced or metastatic ovarian cancer (including EOC). In some embodiments, the cancer is peritoneal cancer. In some such embodiments, the peritoneal cancer is primary peritoneal carcinomatosis (PPC). In some embodiments, the cancer is fallopian tube cancer (FTC).

In another aspect, the invention provides a method of treating breast cancer in a subject in need thereof comprising administering to the subject a therapeutically effective amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A). In some embodiments, the breast cancer is HR+/HER2- breast cancer. In some such embodiments, the breast cancer is HR+/HER2- advanced or metastatic breast cancer. In some embodiments, the breast cancer is triple negative breast cancer (TNBC). In some such embodiments, the TNBC is locally recurrent, advanced, or metastatic TNBC. In some embodiments of the foregoing, the breast cancer is HR+/HER2- breast cancer or TNBC, which may be advanced or metastatic, and the subject is a woman of any menopausal status or a man.

In some embodiments of the foregoing methods, (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) is administered as a single agent.

In other embodiments, the foregoing methods further comprise administering to the subject an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) in combination with an amount of an additional anti-cancer agent, wherein the amounts of COMPOUND A and the additional anti-cancer agent together are effective in treating cancer. In some embodiments wherein the cancer is breast cancer, the additional anti-cancer agent is an endocrine therapeutic agent. In some such embodiments, the endocrine therapeutic agent is an aromatase inhibitor, a SERD, or a SERM. In preferred embodiments, the endocrine therapeutic agent is letrozole or fulvestrant.

In another aspect, the invention provides (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for use in treating cancer. In some embodiments the invention provides COMPOUND A for use in treating cancer, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

In another aspect, the invention provides (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for use in treating lung cancer. In some embodiments of this aspect, the lung cancer is small cell lung cancer (SCLC). In some such embodiments the SCLC is RB-negative. In some embodiments of this aspect, the lung cancer is non-small cell lung cancer (NSCLC). In some embodiments of this aspect, the lung cancer is advanced or metastatic lung cancer. In some such embodiments, the lung cancer is advanced or metastatic SCLC. In some such embodiments, the lung cancer is advanced or metastatic NSCLC.

In another aspect, the invention provides (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for use in treating ovarian cancer, peritoneal cancer, or fallopian tube cancer. In some embodiments, the invention provides COMPOUND A for use in treating ovarian cancer. In some such embodiments, the ovarian cancer is epithelial ovarian cancer (EOC). In some such embodiments, the ovarian cancer is advanced or metastatic ovarian cancer (including EOC). In some such embodiments, the ovarian cancer is platinum resistant ovarian cancer (including EOC). In some such embodiments, the ovarian cancer is platinum resistant advanced or metastatic ovarian cancer (including EOC). In some embodiments the invention provides COMPOUND A for use in treating peritoneal cancer. In some such embodiments, the peritoneal cancer is primary peritoneal carcinomatosis (PPC). In some embodiments the invention provides COMPOUND A for use in treating the fallopian tube cancer (FTC).

In another aspect, the invention provides (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for use in treating breast cancer. In some embodiments, the breast cancer is HR+/HER2- breast cancer. In some such embodiments, the breast cancer is HR+/HER2- advanced or metastatic breast cancer. In some embodiments, the breast cancer is triple negative breast cancer (TNBC). In some such embodiments, the TNBC is locally recurrent, advanced, or metastatic TNBC. In some embodiments of the foregoing, the breast cancer is HR+/HER2- breast cancer or TNBC, which may be advanced or metastatic, and the subject is a woman of any menopausal status or a man.

In some embodiments of the foregoing, (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) is used as a single agent.

In other embodiments of the foregoing, the invention provides (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for use in treating cancer, wherein COMPOUND A is administered in combination with an additional anti-cancer agent. In some such embodiments, COMPOUND A and the additional anti-cancer agent together are effective in treating cancer. In some embodiments wherein the cancer is breast cancer, the additional anti-cancer agent is an endocrine therapeutic agent. In some such embodiments, the endocrine therapeutic agent is an aromatase inhibitor, a SERD, or a SERM. In some such embodiments, the endocrine therapeutic agent is letrozole. In other such embodiments, the endocrine therapeutic agent is fulvestrant.

In another aspect, the invention provides use of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for treating cancer. In some embodiments the invention provides COMPOUND A for use in treating cancer, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

In another aspect, the invention provides use of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for treating lung cancer. In some embodiments of this aspect, the lung cancer is small cell lung cancer (SCLC). In some such embodiments the SCLC is RB-negative. In some embodiments of this aspect, the lung cancer is non-small cell lung cancer (NSCLC). In some embodiments of this aspect, the lung cancer is advanced or metastatic lung cancer. In some such embodiments, the lung cancer is advanced or metastatic SCLC. In some such embodiments, the lung cancer is advanced or metastatic NSCLC.

In another aspect, the invention provides use of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for treating ovarian cancer, peritoneal cancer, or fallopian tube cancer. In some embodiments, the invention provides use of COMPOUND A for treating ovarian cancer. In some such embodiments, the ovarian cancer is epithelial ovarian cancer (EOC). In some such embodiments, the ovarian cancer is advanced or metastatic ovarian cancer (including EOC). In some such embodiments, the ovarian cancer is platinum resistant ovarian cancer (including EOC). In some such embodiments, the ovarian cancer is platinum resistant advanced or metastatic ovarian cancer (including EOC). In some embodiments the invention provides use of COMPOUND A for treating peritoneal cancer. In some such embodiments, the peritoneal cancer is primary peritoneal carcinomatosis (PPC). In some embodiments the invention provides use of COMPOUND A for treating the fallopian tube cancer (FTC).

In another aspect, the invention provides use of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for treating breast cancer. In some embodiments, the breast cancer is HR+/HER2- breast cancer. In some such embodiments, the breast cancer is HR+/HER2- advanced or metastatic breast cancer. In some embodiments, the breast cancer is triple negative breast cancer (TNBC). In some such embodiments, the TNBC is locally recurrent, advanced, or metastatic TNBC. In some embodiments of the foregoing, the breast cancer is HR+/HER2- breast cancer or TNBC, which may be advanced or metastatic, and the subject is a woman of any menopausal status or a man.

In some embodiments of the foregoing, the invention provides use of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) as a single agent.

In other embodiments of the foregoing, the invention provides use of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) for treating cancer, wherein COMPOUND A is administered in combination with an additional anti-cancer agent. In some such embodiments, use of COMPOUND A and the additional anti-cancer agent together are effective in treating cancer. In some embodiments wherein the cancer is breast cancer, the additional anti-cancer agent is an endocrine therapeutic agent. In some such embodiments, the endocrine therapeutic agent is an aromatase inhibitor, a SERD, or a SERM. In some such embodiments, the endocrine therapeutic agent is letrozole. In other such embodiments, the endocrine therapeutic agent is fulvestrant.

The invention further provides methods and uses comprising a pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient as a single agent, optionally in combination with an additional anti-cancer agent, as further described below.

In one aspect, the invention provides a method of treating cancer in a subject in need thereof comprising administering to the subject a therapeutically effective amount of a pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient. In some embodiments the invention provides a method of treating cancer comprising administering to the subject a therapeutically effective amount of a pharmaceutical composition comprising COMPOUND A and a pharmaceutically acceptable excipient, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

In another aspect, the invention provides a method of treating lung cancer in a subject in need thereof comprising administering to the subject a therapeutically effective amount of a pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient. In some embodiments of this aspect, the lung cancer is small cell lung cancer (SCLC). In some such embodiments the SCLC is RB-negative. In some embodiments of this aspect, the lung cancer is non-small cell lung cancer (NSCLC). In some embodiments of this aspect, the lung cancer is advanced or metastatic lung cancer. In some such embodiments, the lung cancer is advanced or metastatic SCLC. In some such embodiments, the lung cancer is advanced or metastatic NSCLC.

In another aspect, the invention provides a method of treating ovarian cancer, peritoneal cancer, or fallopian tube cancer in a subject in need thereof comprising administering to the subject a therapeutically effective amount of a pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient. In some embodiments, the cancer is ovarian cancer. In some such embodiments, the ovarian cancer is epithelial ovarian cancer (EOC). In some such embodiments, the ovarian cancer is advanced or metastatic ovarian cancer (including EOC). In some such embodiments, the ovarian cancer is platinum resistant ovarian cancer (including EOC). In some such embodiments, the ovarian cancer is platinum resistant advanced or metastatic ovarian cancer (including EOC). In some embodiments, the cancer is peritoneal cancer. In some such embodiments, the peritoneal cancer is primary peritoneal carcinomatosis (PPC). In some embodiments, the cancer is fallopian tube cancer (FTC).

In another aspect, the invention provides a method of treating breast cancer in a subject in need thereof comprising administering to the subject a therapeutically effective amount of a pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient. In some embodiments, the breast cancer is HR+/HER2- breast cancer. In some such embodiments, the breast cancer is HR+/HER2- advanced or metastatic breast cancer. In some embodiments, the breast cancer is triple negative breast cancer (TNBC). In some such embodiments, the TNBC is locally recurrent, advanced, or metastatic TNBC. In some embodiments of the foregoing, the breast cancer is HR+/HER2- breast cancer or TNBC, which may be advanced or metastatic, and the subject is a woman of any menopausal status or a man.

In some embodiments of the foregoing methods, the pharmaceutical composition comprising COMPOUND A and a pharmaceutically acceptable excipient is administered as a single agent.

In other embodiments of the foregoing methods, the method further comprises administering to the subject an amount of an additional anti-cancer agent, wherein the amount of the pharmaceutical composition comprising COMPOUND A and a pharmaceutically acceptable excipient and the amount of the additional anti-cancer agent together are effective in treating cancer. In some embodiments wherein the cancer is breast cancer, the additional anti-cancer agent is an endocrine therapeutic agent. In some such embodiments, the endocrine therapeutic agent is an aromatase inhibitor, a SERD, or a SERM. In some such embodiments, the endocrine therapeutic agent is letrozole. In other such embodiments, the endocrine therapeutic agent is fulvestrant.

In another aspect, the invention provides a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A) and a pharmaceutically acceptable excipient for use in treating cancer. In some embodiments the invention provides a pharmaceutical composition comprising COMPOUND A and a pharmaceutically acceptable excipient for use in treating cancer, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

In another aspect, the invention provides a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A) and a pharmaceutically acceptable excipient for use in treating lung cancer. In some embodiments of this aspect, the lung cancer is small cell lung cancer (SCLC). In some such embodiments the SCLC is RB-negative. In some embodiments of this aspect, the lung cancer is non-small cell lung cancer (NSCLC). In some embodiments of this aspect, the lung cancer is advanced or metastatic lung cancer. In some such embodiments, the lung cancer is advanced or metastatic SCLC. In some such embodiments, the lung cancer is advanced or metastatic NSCLC.

In another aspect, the invention provides a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A) and a pharmaceutically acceptable excipient for use in treating ovarian cancer, peritoneal cancer, or fallopian tube cancer. In some embodiments, the invention provides a pharmaceutical composition comprising COMPOUND A and a pharmaceutically acceptable excipient for use in treating ovarian cancer. In some such embodiments, the ovarian cancer is epithelial ovarian cancer (EOC). In some such embodiments, the ovarian cancer is advanced or metastatic ovarian cancer (including EOC). In some such embodiments, the ovarian cancer is platinum resistant ovarian cancer (including EOC). In some such embodiments, the ovarian cancer is platinum resistant advanced or metastatic ovarian cancer (including EOC). In some embodiments the invention provides a pharmaceutical composition comprising COMPOUND A and a pharmaceutically acceptable excipient for use in treating peritoneal cancer. In some such embodiments, the peritoneal cancer is primary peritoneal carcinomatosis (PPC). In some embodiments the invention provides a pharmaceutical composition comprising COMPOUND A and a pharmaceutically acceptable excipient for use in treating the fallopian tube cancer (FTC).

In another aspect, the invention provides a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A) and a pharmaceutically acceptable excipient for use in treating breast cancer. In some embodiments, the breast cancer is HR+/HER2- breast cancer. In some such embodiments, the breast cancer is HR+/HER2- advanced or metastatic breast cancer. In some embodiments, the breast cancer is triple negative breast cancer (TNBC). In some such embodiments, the TNBC is locally recurrent, advanced, or metastatic TNBC. In some embodiments of the foregoing, the breast cancer is HR+/HER2- breast cancer or TNBC, which may be advanced or metastatic, and the subject is a woman of any menopausal status or a man.

In some embodiments of the foregoing, the invention provides the pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient for use as a single agent.

In other embodiments of the foregoing, the invention provides a pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient for use in treating cancer, wherein the pharmaceutical composition comprising COMPOUND A and a pharmaceutically acceptable excipient is administered in combination with an additional anti-cancer agent. In some such embodiments, the pharmaceutical composition comprising COMPOUND A and a pharmaceutically acceptable excipient and the additional anti-cancer agent together are effective in treating cancer. In some embodiments wherein the cancer is breast cancer, the additional anti-cancer agent is an endocrine therapeutic agent. In some such embodiments, the endocrine therapeutic agent is an aromatase inhibitor, a SERD, or a SERM. In some such embodiments, the endocrine therapeutic agent is letrozole. In other such embodiments, the endocrine therapeutic agent is fulvestrant.

In another aspect, the invention provides use of a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A) and a pharmaceutically acceptable excipient for treating cancer. In some embodiments the invention provides use of a pharmaceutical composition comprising COMPOUND A and a pharmaceutically acceptable excipient for treating cancer, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

In another aspect, the invention provides use of a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A) and a pharmaceutically acceptable excipient for treating lung cancer. In some embodiments of this aspect, the lung cancer is small cell lung cancer (SCLC). In some such embodiments the SCLC is RB-negative. In some embodiments of this aspect, the lung cancer is non-small cell lung cancer (NSCLC). In some embodiments of this aspect, the lung cancer is advanced or metastatic lung cancer. In some such embodiments, the lung cancer is advanced or metastatic SCLC. In some such embodiments, the lung cancer is advanced or metastatic NSCLC.

In another aspect, the invention provides use of a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A) and a pharmaceutically acceptable excipient for treating ovarian cancer, peritoneal cancer, or fallopian tube cancer. In some embodiments, the invention provides use of a pharmaceutical composition comprising COMPOUND A and a pharmaceutically acceptable excipient for treating ovarian cancer. In some such embodiments, the ovarian cancer is epithelial ovarian cancer (EOC). In some such embodiments, the ovarian cancer is advanced or metastatic ovarian cancer (including EOC). In some such embodiments, the ovarian cancer is platinum resistant ovarian cancer (including EOC). In some such embodiments, the ovarian cancer is platinum resistant advanced or metastatic ovarian cancer (including EOC). In some embodiments the invention provides use of a pharmaceutical composition comprising COMPOUND A and a pharmaceutically acceptable excipient for treating peritoneal cancer. In some such embodiments, the peritoneal cancer is primary peritoneal carcinomatosis (PPC). In some embodiments the invention provides use of a pharmaceutical composition comprising COMPOUND A and a pharmaceutically acceptable excipient for treating the fallopian tube cancer (FTC).

In another aspect, the invention provides use of a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate (COMPOUND A) and a pharmaceutically acceptable excipient for treating breast cancer. In some embodiments, the breast cancer is HR+/HER2- breast cancer. In some such embodiments, the breast cancer is HR+/HER2- advanced or metastatic breast cancer. In some embodiments, the breast cancer is triple negative breast cancer (TNBC). In some such embodiments, the TNBC is locally recurrent, advanced, or metastatic TNBC. In some embodiments of the foregoing, the breast cancer is HR+/HER2- breast cancer or TNBC, which may be advanced or metastatic, and the subject is a woman of any menopausal status or a man.

In some embodiments of the foregoing, the invention provides use of the pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient as a single agent.

In other embodiments of the foregoing, the invention provides use of a pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A) and a pharmaceutically acceptable excipient for treating cancer, wherein the pharmaceutical composition comprising COMPOUND A and a pharmaceutically acceptable excipient is administered in combination with an additional anti-cancer agent. In some such embodiments, use of the pharmaceutical composition comprising COMPOUND A and a pharmaceutically acceptable excipient and the additional anti-cancer agent together are effective in treating cancer. In some embodiments wherein the cancer is breast cancer, the additional anti-cancer agent is an endocrine therapeutic agent. In some such embodiments, the endocrine therapeutic agent is an aromatase inhibitor, a SERD, or a SERM. In some such embodiments, the endocrine therapeutic agent is letrozole. In other such embodiments, the endocrine therapeutic agent is fulvestrant.

### Dosage Forms and Regimens

Each therapeutic agent of the methods and combination therapies of the present invention may be administered either alone, or in a medicament (also referred to herein as a pharmaceutical composition) which comprises the therapeutic agent and one or more pharmaceutically acceptable carriers, excipients, or diluents, according to pharmaceutical practice.

As used herein, the terms “combination” or “combination therapy” refer to the administration of each therapeutic agent of the combination therapy of the invention, either alone or in the form of a pharmaceutical composition or medicament, either sequentially, concurrently, or simultaneously.

As used herein, the term “sequential” or “sequentially” refers to the administration of each therapeutic agent of the combination therapy of the invention, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

As used herein, the term “concurrently” refers to the administration of each therapeutic agent in the combination therapy of the invention, either alone or in separate medicaments, wherein the second therapeutic agent is administered immediately after the first therapeutic agent, but that the therapeutic agents can be administered in any order. In a preferred embodiment the therapeutic agents are administered concurrently.

As used herein, the term “simultaneous” refers to the administration of each therapeutic agent of the combination therapy of the invention in the same medicament.

As will be understood by those skilled in the art, the combination therapy may be usefully administered to a subject during different stages of their treatment.

In some embodiments of each of the methods, combinations and uses herein, the combination therapy is administered to a subject who is previously untreated, i.e. is treatment naïve.

In some embodiments of each of the methods, combinations and uses herein, the combination therapy is administered to a subject who has failed to achieve a sustained response after a prior therapy with a biotherapeutic or chemotherapeutic agent, i.e. is treatment experienced.

In some embodiments of each of the methods, combinations and uses herein, the combination therapy may be administered prior to of following surgery to remove a tumor and / or may be used prior to, during or after radiation therapy, and / or may be used prior to, during or after chemotherapy.

In some embodiments of each of the methods, combinations and uses herein, the invention relates to neoadjuvant therapy, adjuvant therapy, first-line therapy, second-line therapy, or third-line or later therapy, in each case for treating cancer as further described herein. In each of the foregoing embodiments, the cancer may be localized, advanced or metastatic, and the intervention may occur at point along the disease continuum (i.e., at any stage of the cancer).

The efficacy of combinations described herein in certain tumors may be enhanced by combination with other approved or experimental cancer therapies, e.g., radiation, surgery, chemotherapeutic agents, targeted therapies, agents that inhibit other signaling pathways that are dysregulated in tumors, and other immune enhancing agents, such as PD-1 or PD-L1 antagonists and the like. The methods, combinations and uses of the current invention may further comprise one or more additional anti-cancer agents.

Administration of combinations of the invention may be affected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a therapeutic agent of the combination therapy of the present invention may be administered as a single bolus, as several divided doses administered over time, or the dose may be proportionally reduced or increased as indicated by the exigencies of the therapeutic situation. It may be particularly advantageous to formulate a therapeutic agent in a dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention may be dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose may be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, taking into consideration factors such as the severity of the disorder or condition, the rate of administration, the disposition of the compound and the discretion of the prescribing physician. The dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

In some embodiments, at least one of the therapeutic agents in the combination therapy is administered using the same dosage regimen (dose, frequency, and duration of treatment) that is typically employed when the agent is used as a monotherapy for treating the same cancer. In other embodiments, the subject received a lower total amount of at least one of the therapeutic agents in the combination therapy than when the same agent is used as a monotherapy, for example a lower dose of therapeutic agent, a reduced frequency of dosing and / or a shorter duration of dosing.

An effective dosage of a small molecule inhibitor is typically in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day, and more preferably from about 0.02 to about 1.0 g/day. In some instances, dosage levels at the lower limit of the aforesaid range may be more than adequate, while in other cases still larger doses may be employed without causing any harmful side effect, provided that such larger doses are first divided into several small doses for administration throughout the day. The dosage may be administered as a single dose (QD), or optionally may be subdivided into smaller doses, suitable for BID (twice daily), TID (three times daily) or QID (four times daily) administration.

In some embodiments, the CDK2 inhibitor of Formula (I), or a pharmaceutically acceptable salt or solvate thereof, is administered at a daily dosage of from about 1 mg to about 1000 mg per day. In some embodiments, the compound of Formula (I) or a pharmaceutically acceptable salt or solvate thereof, is administered at a daily dosage from about 10 mg to about 500 mg per day, and in some embodiments, it is administered at a dosage of from about 25 mg to about 300 mg per day. In some embodiments it is administered at dosages of about 1, 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 260, 270, 275, 280, 290, 300, 325, 350, 375, 400, 425, 450, 475 or 500 mg on a QD, BID, TID or QID schedule.

In certain embodiments, the CDK4/6 inhibitor is palbociclib, or a pharmaceutically acceptable salt or solvate thereof, which is administered orally at a daily dosage of about 25 mg to about 125 mg per day, and sometimes at a dosage of 25 mg, 50 mg, 75 mg, 100 mg, or 125 mg per day. In other embodiments, the CDK4/6 inhibitor is ribociclib, or a pharmaceutically acceptable salt or solvate thereof, which is administered orally at a daily dosage of about 200 mg to about 600 mg per day; or the CDK4/6 inhibitor is abemaciclib, or a pharmaceutically acceptable salt or solvate thereof, which is administered orally at a daily dosage of about 150 mg to about 400 mg per day.

In some embodiments, the endocrine therapeutic agent is letrozole, which may be administered orally at a dose of 2.5 mg daily. In some embodiments, the endocrine therapeutic agent is fulvestrant, which may be administered intramuscularly in one or two injections at a dose of 250 mg or 500 mg, respectively, on Days 1, 15, 29, of the first month and then once monthly thereafter.

Repetition of the administration or dosing regimens, or adjustment of the administration or dosing regimen for each compound included in the combination therapy may be conducted as necessary to achieve the desired treatment. An “intermittent dosing schedule” as used herein refers to an administration or dosing regimen that includes a period of dose interruption, e.g. days off treatment. Repetition of 14 or 21 day treatment cycles with a 7 day treatment interruption between the treatment cycles is an example of an intermittent dosing schedule. Such schedules, with 2 or 3 weeks on treatment and 1 week off treatment, are sometimes referred to as a 2/1-week or 3/1-week treatment cycle, respectively. Alternatively, intermittent dosing may comprise a 7 day treatment cycle, with 5 days on treatment and 2 days off treatment.

A “continuous dosing schedule” as used herein is an administration or dosing regimen without dose interruptions, e.g. without days off treatment. Repetition of 21 or 28 day treatment cycles without dose interruptions between the treatment cycles is an example of a continuous dosing schedule.

In some embodiments, the compound of Formula (I) and the CDK4/6 inhibitor are administered in an intermittent dosing schedule. In other embodiments, the compound of Formula (I) and the CDK4/6 are administered in a continuous dosing schedule.

In still other embodiments, one of the compound of Formula (I) and the CDK4/6 is administered in an intermittent dosing schedule (e.g., a 2/1-week or 3/1-week schedule) and the other is administered in a continuous dosing schedule. In some such embodiments, the compound of Formula (I) is administered in an intermittent dosing schedule and the CDK4/6 inhibitor is administered in a continuous dosing schedule. In other such embodiments, the compound of Formula (I) is administered in a continuous dosing schedule and the CDK4/6 inhibitor is administered in an intermittent dosing schedule.

In some embodiments of the present invention, the compound of Formula (I) and the CDK4/6 inhibitor are dosed in amounts which together are effective in treating the cancer.

In some embodiments of the present invention, the compound of Formula (I) and the CDK4/6 inhibitor are dosed in amounts which together are synergistic.

In some embodiments of the present invention, the compound of Formula (I) and the CDK4/6 inhibitor are dosed in amounts which together are additive.

### Pharmaceutical Compositions and Routes of Administration

A “pharmaceutical composition” refers to a mixture of one or more of the therapeutic agents described herein, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients.

As used herein, a “pharmaceutically acceptable carrier” refers to a carrier or diluent that does not cause significant irritation to an organism and does not abrogate the biological activity and properties of the active compound or therapeutic agent.

The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form.

Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream, or for rectal administration as a suppository.

Exemplary parenteral administration forms include solutions or suspensions of an active compound in a sterile aqueous solution, for example, aqueous propylene glycol or dextrose solutions. Such dosage forms may be suitably buffered, if desired.

The pharmaceutical composition may be in unit dosage forms suitable for single administration of precise amounts.

Pharmaceutical compositions suitable for the delivery of the therapeutic agents of the combination therapies of the present invention, and methods for their preparation will be readily apparent to those skilled in the art. Such compositions and methods for their preparation may be found, for example, in ‘Remington’s Pharmaceutical Sciences’, 19th Edition (Mack Publishing Company, 1995), the disclosure of which is incorporated herein by reference in its entirety.

Therapeutic agents of the combination therapies of the invention may be administered orally. Oral administration may involve swallowing, so that the therapeutic agent enters the gastrointestinal tract, or buccal or sublingual administration may be employed by which the therapeutic agent enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups, and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Therapeutic agents of the combination therapies of the present invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, the therapeutic agent may make up from 1 wt% to 80 wt% of the dosage form, more typically from 5 wt% to 60 wt% of the dosage form. In addition to the active agent, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant may comprise from 1 wt% to 25 wt%, preferably from 5 wt% to 20 wt% of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch, and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt% to 5 wt% of the tablet, and glidants typically from 0.2 wt% to 1 wt% of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt% to 10 wt%, preferably from 0.5 wt% to 3 wt% of the tablet.

Other conventional ingredients include anti-oxidants, colorants, flavoring agents, preservatives, and taste-masking agents.

Exemplary tablets may contain up to about 80 wt% active agent, from about 10 wt% to about 90 wt% binder, from about 0 wt% to about 85 wt% diluent, from about 2 wt% to about 10 wt% disintegrant, and from about 0.25 wt% to about 10 wt% lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles may be found in Verma et al., Pharmaceutical Technology On-line, 25(2), 1-14 (2001). The use of chewing gum to achieve controlled release is described in WO 2000/035298. The disclosures of these references are incorporated herein by reference in their entireties.

The kits described herein may be particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid. The kit may further comprise other materials that may be useful in administering the medicaments, such as diluents, filters, IV bags and lines, needles and syringes, and the like.

### Additional Anti-Cancer Agents

The methods, combinations and uses of the present invention may additionally comprise one or more additional anti-cancer agents, such as the anti-angiogenesis agents, signal transduction inhibitors or antineoplastic agents described below, wherein the amounts are together effective in treating cancer. In some embodiments, the methods, combinations and uses of the present invention the additional anti-cancer agents may comprise a palliative care agent. Additional anti-cancer agents may include small molecules therapeutics and pharmaceutically acceptable salts or solvates thereof, therapeutic antibodies, antibody-drug conjugates (ADCs), hetero-bifunctional protein degraders (e.g., proteolysis targeting chimeras or PROTACs), or antisense molecules.

In some embodiments, the methods, combinations and uses of the present invention further comprise one or more additional anti-cancer agents selected from the following:

Anti-angiogenesis agents include, for example, VEGF inhibitors, VEGFR inhibitors, TIE-2 inhibitors, PDGFR inhibitors, angiopoetin inhibitors, PKCβ inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors.

Signal transduction inhibitors include, for example, kinase inhibitors (e.g., inhibitors of tyrosine kinases, serine/threonine kinases or cyclin dependent kinases), proteasome inhibitors, PI3K/AKT/mTOR pathway inhibitors, Phosphoinositide 3-kinase (PI3K) inhibitors, isocitrate dehydrogenase 1 and 2 (IDH1 and IDH2) inhibitors, B-cell lymphoma 2 (BCL2) inhibitors, neurotrophin receptor kinase (NTRK) inhibitors, Rearranged during Transfection (RET) inhibitors, Notch inhibitors, PARP inhibitors, Hedgehog pathway inhibitors, and selective inhibitors of nuclear export (SINE).

Examples of signal transduction inhibitors inhibitors include, but are not limited to: acalabrutinib, afatinib, alectinib, alpelisib, axitinib, binimetinib, bortezomib, bosutinib, brigatinib, cabozantinib, carfilzomib, ceritinib, cobimetinib, copanlisib, crizotinib, dabrafenib, dacomitinib, dasatinib, duvelisib, enasidenib, encorafenib, entrectinib, erlotinib, gefitinib, gilteritinib, glasdegib, ibrutinib, idelalisib, imatinib, ipatasertib, ivosidenib, ixazomib, lapatinib, larotrectinib, lenvatinib, lorlatinib, midostaurin, neratinib, nilotinib, niraparib, olaparib, osimertinib, pazopanib, ponatinib, regorafenib, rucaparib, ruxolitinib, sonidegib, sorafenib, sunitinib, talazoparib, trametinib, vandetanib, vemurafenib, venetoclax, and vismodegib, or pharmaceutically acceptable salts and solvates thereof.

Antineoplastic agents include, for example, alkylating agents, platinum coordination complexes, cytotoxic antibiotics, antimetabolies, biologic response modifiers, histone deacetylate (HDAC) inhibitors, hormonal agents, monoclonal antibodies, growth factor inhibitors, taxanes, topoisomerase inhibitors, Vinca alkaloids and miscellaneous agents.

Alkylating agents include: altretamine, bendamustine, busulfan, carmustine, chlorambucil, cyclophosphamide, dacarbazine, ifosfamide, lomustine, mechlorethamine, melphalan, procarbazine, streptozocin, temozolomide, thiotepa, and trabectedin.

Platinum coordination complexes include: carboplatin, cisplatin, and oxaliplatin.

Cytotoxic antibiotics include: bleomycin, dactinomycin, daunorubicin, doxorubicin, epirubicin, idarubicin, mitomycin, mitoxantrone, plicamycin, and valrubicin.

Antimetabolites include: antifolates, such as methotrexate, pemetrexed, pralatrexate, and trimetrexate; purine analogues, such as azathioprine, cladribine, fludarabine, mercaptopurine, and thioguanine; and pyrimidine analogues such as azacitidine, capecitabine, cytarabine, decitabine, floxuridine, fluorouracil, gemcitabine, and trifluridine/tipracil.

Biologic response modifiers include: aldesleukin (IL-2), denileukin diftitox, and interferon gamma.

Histone deacetylase inhibitors include belinostat, panobinostat, romidepsin, and vorinostat.

Endocrine therapeutic agents (i.e., hormonal therapy agents) include antiandrogens, antiestrogens, gonadotropin releasing hormone (GnRH) analogues and peptide hormones. Examples of antiestrogens include: aromatase inhibitors, such as letrozole, anastrozole, and exemestane; SERDs, such as fulvestrant, elacestrant (RAD-1901, Radius Health/Menarini), amcenestrant (SAR439859, Sanofi), giredestrant (GDC9545, Roche), RG6171 (Roche), camizestrant (AZD9833, AstraZeneca), AZD9496 (AstraZeneca), rintodestrant (G1 Therapeutics), ZN-c5 (Zentalis), LSZ102 (Novartis), D-0502 (Inventisbio), LY3484356 (Eli Lilly), SHR9549 (Jiansu Hengrui Medicine); and SERMs, such as tamoxifen, raloxifene, toremifene, lasofoxifene, bazedoxifene, afimoxifene. Examples of GnRH analogues include: degarelix, goserelin, histrelin, leuprolide, and triptorelin. Examples of peptide hormones include: lanreotide, octreotide, and pasireotide. Examples of antiandrogens include: abiraterone, apalutamide, bicalutamide, cyproterone, enzalutamide, flutamide, and nilutamide, and pharmaceutically acceptable salts and solvates thereof.

Monoclonal antibodies include: alemtuzumab, atezolizumab, avelumab, bevacizumab, blinatumomab, brentuximab, cemiplimab, cetuximab, daratumumab, dinutuximab, durvalumab, elotuzumab, gemtuzumab, inotuzumab ozogamicin, ipilimumab, mogamulizumab, moxetumomab pasudotox, necitumumab, nivolumab, ofatumumab, olaratumab, panitumumab, pembrolizumab, pertuzumab, ramucirumab, rituximab, tositumomab, and trastuzumab.

Taxanes include: cabazitaxel, docetaxel, paclitaxel and paclitaxel albumin-stabilized nanoparticle formulation.

Topoisomerase inhibitors include: etoposide, irinotecan, teniposide, and topotecan.

Vinca alkaloids include: vinblastine, vincristine, and vinorelbine, and pharmaceutically acceptable salts thereof.

Miscellaneous antineoplastic agents include: asparaginase (pegaspargase), bexarotene, eribulin, everolimus, hydroxyurea, ixabepilone, lenalidomide, mitotane, omacetaxine, pomalidomide, tagraxofusp, telotristat, temsirolimus, thalidomide, and venetoclax.

In some embodiments, the additional anti-cancer agent is selected from the group consisting of: abiraterone acetate; acalabrutinib; ado-trastuzumab emtansine; afatinib dimaleate; afimoxifene; aldesleukin; alectinib; alemtuzumab; alpelisib; amifostine; anastrozole; apalutamide; aprepitant; arsenic trioxide; asparaginase erwinia chrysanthemi; atezolizumab; avapritinib; avelumab; axicabtagene ciloleucel; axitinib; azacitidine; AZD9833 (AstraZeneca); AZD9496 (AstraZeneca); bazedoxifene; belinostat; bendamustine hydrochloride; bevacizumab; bexarotene; bicalutamide; binimetinib; bleomycin sulfate; blinatumomab; bortezomib; bosutinib; brentuximab vedotin; brigatinib; cabazitaxel; cabozantinib-s-malate; calaspargase pegol-mknl; capecitabine; caplacizumab-yhdp; capmatinib hydrochloride; carboplatin; carfilzomib; carmustine; cemiplimab-rwlc; ceritinib; cetuximab; chlorambucil; cisplatin; cladribine; clofarabine; cobimetinib; copanlisib hydrochloride; crizotinib; cyclophosphamide; cytarabine; D-0502 (Inventisbio); dabrafenib mesylate; dacarbazine; dacomitinib; dactinomycin; daratumumab; daratumumab and hyaluronidase-fihj; darbepoetin alfa; darolutamide; dasatinib; daunorubicin hydrochloride; decitabine; defibrotide sodium; degarelix; denileukin diftitox; denosumab; dexamethasone; dexrazoxane hydrochloride; dinutuximab; docetaxel; doxorubicin hydrochloride; durvalumab; duvelisib; elacestrant; elotuzumab; eltrombopag olamine; emapalumab-lzsg; enasidenib mesylate; encorafenib; enfortumab vedotin-ejfv; entrectinib; enzalutamide; epirubicin hydrochloride; epoetin alfa; erdafitinib; eribulin mesylate; erlotinib hydrochloride; etoposide; etoposide phosphate; everolimus; exemestane; fam-trastuzumab deruxtecan-nxki; fedratinib hydrochloride; filgrastim; fludarabine phosphate; fluorouracil; flutamide; fostamatinib disodium; fulvestrant; gefitinib; gemcitabine hydrochloride; gemtuzumab ozogamicin; gilteritinib fumarate; glasdegib maleate; glucarpidase; goserelin acetate; granisetron; granisetron hydrochloride; hydroxyurea; ibritumomab tiuxetan; ibrutinib; idarubicin hydrochloride; idelalisib; ifosfamide; imatinib mesylate; imiquimod; inotuzumab ozogamicin; interferon alfa-2b recombinant; iobenguane 1-131; ipatasertib; ipilimumab; irinotecan hydrochloride; isatuximab-irfc; ivosidenib; ixabepilone; ixazomib citrate; lanreotide acetate; lapatinib ditosylate; larotrectinib sulfate; lasofoxifene; lenalidomide; lenvatinib mesylate; letrozole; leucovorin calcium; leuprolide acetate; lomustine; lorlatinib; LSZ102 (Novartis); lurbinectedin; LY3484356 (Lilly); megestrol acetate; melphalan; melphalan hydrochloride; mercaptopurine; methotrexate; midostaurin; mitomycin ; mitoxantrone hydrochloride; mogamulizumab-kpkc; moxetumomab pasudotox-tdfk; necitumumab; nelarabine; neratinib maleate; nilotinib; nilutamide; niraparib tosylate monohydrate; nivolumab; obinutuzumab; ofatumumab; olaparib; omacetaxine mepesuccinate; ondansetron hydrochloride; osimertinib mesylate; oxaliplatin; paclitaxel; paclitaxel albumin-stabilized nanoparticle formulation; palifermin; palonosetron hydrochloride; pamidronate disodium; panitumumab; panobinostat; pazopanib hydrochloride; pegaspargase; pegfilgrastim; peginterferon alfa-2b; pembrolizumab; pemetrexed disodium; pemigatinib; pertuzumab; pexidartinib hydrochloride; plerixafor; polatuzumab vedotin-piiq; pomalidomide; ponatinib hydrochloride; pralatrexate; prednisone; procarbazine hydrochloride; propranolol hydrochloride; radium 223 dichloride; raloxifene hydrochloride; ramucirumab; rasburicase; ravulizumab-cwvz; recombinant interferon alfa-2b; regorafenib; RG6171 (Roche); rintodestrant; ripretinib; rituximab; rolapitant hydrochloride; romidepsin; romiplostim; rucaparib camsylate; ruxolitinib phosphate; sacituzumab govitecan-hziy; SAR439859 (Sanofi); selinexor; selpercatinib; selumetinib sulfate; SHR9549 (Jiansu Hengrui Medicine); siltuximab; sipuleucel-t; sonidegib; sorafenib tosylate; tagraxofusp-erzs; talazoparib tosylate; talimogene laherparepvec; tamoxifen citrate; tazemetostat hydrobromide; temozolomide; temsirolimus; thalidomide; thioguanine; thiotepa; tisagenlecleucel; tocilizumab; topotecan hydrochloride; toremifene; trabectedin; trametinib; trastuzumab; trastuzumab and hyaluronidase-oysk; trifluridine and tipiracil hydrochloride; tucatinib; uridine triacetate; valrubicin; vandetanib; vemurafenib; venetoclax; vinblastine sulfate; vincristine sulfate; vinorelbine tartrate; vismodegib; vorinostat; zanubrutinib ; ziv-aflibercept; ZN-c5 (Zentalis); and zoledronic acid; or free base, pharmaceutically acceptable salt, or solvate forms of the foregoing; or combinations thereof.

Preferred embodiments of the invention include embodiments A1-A20 and E1-E97:

A1. A method of treating cancer in a subject in need thereof comprising administering to the subject:
(a) an amount of a compound of Formula (I):

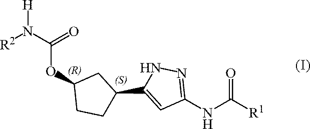

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) an amount of a cyclin dependent kinase 4/6 (CDK4/6) inhibitor;
   wherein the amounts in (a) and (b) together are effective in treating cancer.

A2. The method of embodiment A1, further comprising administering to the subject: (c) an amount of an additional anti-cancer agent; wherein the amounts in (a), (b) and (c) together are effective in treating cancer.

A3. The method of embodiment A1 or A2, wherein the compound of Formula (I) is selected from the group consisting of:
(1R,3S)-3-[3-({[3-(methoxymethyl)-1-methyl-1H-pyrazol-5-yl]carbonyl}amino)-1H-pyrazol-5-yl]cyclopentyl propan-2-ylcarbamate;
(1R,3S)-3-[3-({[2-(methylsulfonyl)phenyl]acetyl}amino)-1H-pyrazol-5-yl]cyclopentyl (2S)-butan-2-ylcarbamate; and
(1R,3S)-3-(3-{[(2-methoxypyridin-4-yl)acetyl]amino}-1H-pyrazol-5-yl)cyclopentyl propylcarbamate;
or a pharmaceutically acceptable salt thereof.

A4. The method of any one of embodiments A1 to A3, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate.

A5. The method of any one of embodiments A1 to A4, wherein the CDK4/6 inhibitor is palbociclib, or a pharmaceutically acceptable salt thereof.

A6. The method of any one of embodiments A1 to A5, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

A7. The method of any one of embodiments A2 to A6, wherein the cancer is hormone receptor positive (HR+), human epidermal growth factor receptor 2 negative (HER2-) breast cancer and the additional anti-cancer agent is an endocrine therapeutic agent selected from the group consisting of an aromatase inhibitor, a SERM and a SERD.

A8. The method of embodiment A7, wherein the endocrine therapeutic agent is letrozole or fulvestrant.

A9. A combination comprising:
(a) a compound of Formula (I):

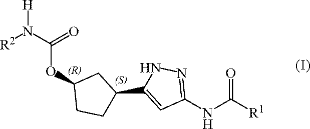

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor;
   wherein the combination of (a) and (b) is effective in treating cancer.

A10. The combination of embodiment A9, further comprising (c) an additional anti-cancer agent; wherein the combination of (a), (b) and (c) is effective in treating cancer.

A11. The combination of embodiment A9 or A10, wherein the compound of Formula (I) is selected from the group consisting of:
(1R,3S)-3-[3-({[3-(methoxymethyl)-1-methyl-1H-pyrazol-5-yl]carbonyl}amino)-1H-pyrazol-5-yl]cyclopentyl propan-2-ylcarbamate;
(1R,3S)-3-[3-({[2-(methylsulfonyl)phenyl]acetyl}amino)-1H-pyrazol-5-yl]cyclopentyl (2S)-butan-2-ylcarbamate; and
(1R,3S)-3-(3-{[(2-methoxypyridin-4-yl)acetyl]amino}-1H-pyrazol-5-yl)cyclopentyl propylcarbamate;
or a pharmaceutically acceptable salt thereof.

A12. The combination of any one of embodiments A9 to A11, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate.

A13. The combination of any one of embodiments A9 to A12, wherein the CDK4/6 inhibitor is palbociclib, or a pharmaceutically acceptable salt thereof.

A14. The combination of any one of embodiments A9 to A13, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

A15. The combination of any one of embodiments A9 to A14, wherein the cancer is HR+, HER2- breast cancer and the additional anti-cancer agent is an endocrine therapeutic agent selected from the group consisting of an aromatase inhibitor, a SERM and a SERD.

A16. The combination of embodiment A15, wherein the endocrine therapeutic agent is letrozole or fulvestrant.

A17. A method of treating cancer in a subject in need thereof comprising administering to the subject:
(a) an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate; and
(b) an amount of palbociclib, or a pharmaceutically acceptable salt thereof;
   wherein the amounts in (a) and (b) together are effective in treating cancer.

A18. A method of treating cancer in a subject in need thereof comprising administering to the subject:
(a) an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate;
(b) an amount of palbociclib, or a pharmaceutically acceptable salt thereof; and
(c) an amount of an endocrine therapeutic agent selected from the group consisting of an aromatase inhibitor, a SERM and a SERD;
   wherein the amounts in (a), (b) and (c) together are effective in treating cancer.

A19. A combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate; and
(b) palbociclib, or a pharmaceutically acceptable salt thereof;
   wherein the combination of (a) and (b) is effective in treating cancer.

A20. A combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate;
(b) palbociclib, or a pharmaceutically acceptable salt thereof; and
(c) an endocrine therapeutic agent selected from the group consisting of an aromatase inhibitor, a SERM and a SERD;
   wherein the combination of (a), (b) and (c) is effective in treating cancer.

E1. A method of treating cancer in a subject in need thereof comprising administering to the subject:
(a) an amount of a compound of Formula (I):

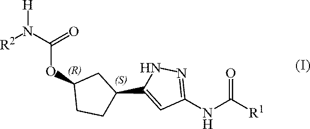

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) an amount of a cyclin dependent kinase 4/6 (CDK4/6) inhibitor;
   wherein the amounts in (a) and (b) together are effective in treating cancer.

E2. The method of embodiment E1, further comprising administering to the subject: (c) an amount of an additional anti-cancer agent; wherein the amounts in (a), (b) and (c) together are effective in treating cancer.

E3. The method of embodiment E1 or E2, wherein the compound of Formula (I) is selected from the group consisting of:
(1R,3S)-3-[3-({[3-(methoxymethyl)-1-methyl-1H-pyrazol-5-yl]carbonyl}amino)-1H-pyrazol-5-yl]cyclopentyl propan-2-ylcarbamate having the structure:

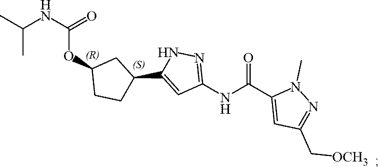

(1R,3S)-3-[3-({[2-(methylsulfonyl)phenyl]acetyl}amino)-1H-pyrazol-5-yl]cyclopentyl (2S)-butan-2-ylcarbamate having the structure:

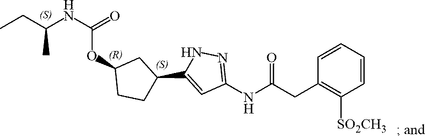

(1R,3S)-3-(3-{[(2-methoxypyridin-4-yl)acetyl]amino}-1H-pyrazol-5-yl)cyclopentyl propylcarbamate, having the structure:

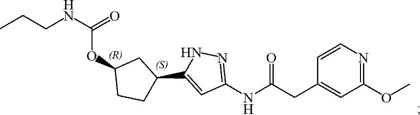

or a pharmaceutically acceptable salt thereof.

E4. The method of any one of embodiments E1 to E3, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate, having the structure:

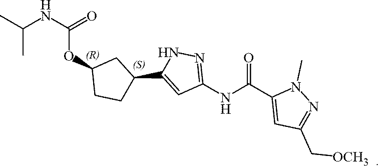

E5. The method of any one of embodiments E1 to E4, wherein the CDK4/6 inhibitor is palbociclib, or a pharmaceutically acceptable salt thereof.

E6. The method of any one of embodiments E1 to E5, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E7. The method of embodiment E6, wherein the cancer is breast cancer.

E8. The method of embodiment E7, wherein the breast cancer is hormone receptor positive (HR+), human epidermal growth factor receptor 2 negative (HER2-) breast cancer.

E9. The method of embodiment E7, wherein the breast cancer is triple negative breast cancer (TNBC).

E10. The method of embodiment E6, wherein the cancer is lung cancer.

E11. The method of embodiment E10, wherein the lung cancer is small cell lung cancer (SCLC).

E12. The method of embodiment E10, wherein the lung cancer is non-small cell lung cancer (NSCLC).

E13. The method of embodiment E6, wherein the cancer is ovarian cancer, peritoneal cancer, or fallopian tube cancer.

E14. The method of embodiment E13, wherein the cancer is epithelial ovarian cancer (EOC), primary peritoneal carcinomatosis (PPC), or fallopian tube cancer (FTC).

E15. The method of embodiment E13 or E14, wherein the ovarian cancer is persistent, refractory or recurrent ovarian cancer.

E16. The method of any one of embodiments E13 to E15, wherein the ovarian cancer is platinum resistant ovarian cancer.

E17. The method of any one of embodiments E1 to E16, wherein the cancer is advanced or metastatic cancer.

E18. The method of any one of embodiments E1 to E17, wherein the cancer is characterized by amplification or overexpression of cyclin E1 (CCNE1) and/or cyclin E2 (CCNE2).

E19. The method of any one of embodiments E1 to E18, wherein the compound of Formula (I) and the CDK4/6 inhibitor are administered sequentially, simultaneously or concurrently.

E20. The method of any one of embodiments E2 to E8, wherein the cancer is HR+/HER2- breast cancer and the additional anti-cancer agent is an endocrine therapeutic agent selected from the group consisting of an aromatase inhibitor, a SERM and a SERD.

E21. The method of embodiment E20, wherein the endocrine therapeutic agent is letrozole.

E22. The method of embodiment E20, wherein the endocrine therapeutic agent is fulvestrant.

E23. A combination comprising:
(a) a compound of Formula (I):

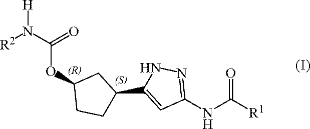

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor;
   wherein the combination of (a) and (b) is effective in treating cancer.

E24. The combination of embodiment E23, further comprising (c) an additional anti-cancer agent; wherein the combination of (a), (b) and (c) is effective in treating cancer.

E25. The combination of embodiment E23 or E24, wherein the compound of Formula (I) is selected from the group consisting of:
(1R,3S)-3-[3-({[3-(methoxymethyl)-1-methyl-1H-pyrazol-5-yl]carbonyl}amino)-1H-pyrazol-5-yl]cyclopentyl propan-2-ylcarbamate, having the structure:

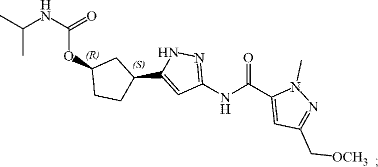

(1R,3S)-3-[3-({[2-(methylsulfonyl)phenyl]acetyl}amino)-1H-pyrazol-5-yl]cyclopentyl (2S)-butan-2-ylcarbamate, having the structure:

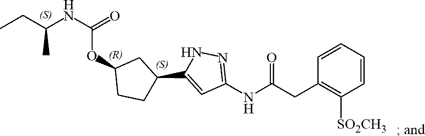

(1R,3S)-3-(3-{[(2-methoxypyridin-4-yl)acetyl]amino}-1H-pyrazol-5-yl)cyclopentyl propylcarbamate, having the structure:

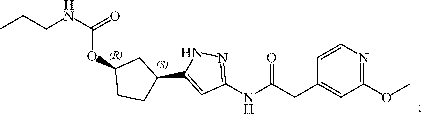

or a pharmaceutically acceptable salt thereof.

E26. The combination of any one of embodiments E23 to E25, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate, having the structure:

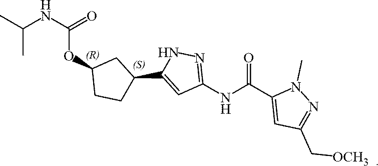

E27. The combination of any one of embodiments E23 to E26, wherein the CDK4/6 inhibitor is palbociclib, or a pharmaceutically acceptable salt thereof.

E28. The combination of any one of embodiments E23 to E27, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E29. The combination of embodiment E28, wherein the cancer is breast cancer.

E30. The combination of embodiment E29, wherein the breast cancer is hormone receptor positive (HR+), human epidermal growth factor receptor 2 negative (HER2-) breast cancer.

E31. The combination of embodiment E29, wherein the breast cancer is triple negative breast cancer (TNBC).

E32. The combination of embodiment E28, wherein the cancer is lung cancer.

E33. The combination of embodiment E32, wherein the lung cancer is small cell lung cancer (SCLC).

E34. The combination of embodiment E32, wherein the lung cancer is non-small cell lung cancer (NSCLC).

E35. The combination of embodiment E28, wherein the cancer is ovarian cancer, peritoneal cancer, or fallopian tube cancer.

E36. The combination of embodiment E35, wherein the cancer is epithelial ovarian cancer (EOC), primary peritoneal carcinomatosis (PPC), or fallopian tube cancer (FTC).

E37. The combination of embodiment E35 or E36, wherein the ovarian cancer is persistent, refractory or recurrent ovarian cancer.

E38. The combination of any one of embodiments E33 to E37, wherein the ovarian cancer is platinum resistant ovarian cancer.

E39. The combination of any one of embodiments E23 to E38, wherein the cancer is advanced or metastatic cancer.

E40. The combination of any one of embodiments E23 to E39, wherein the cancer is characterized by amplification or overexpression of cyclin E1 (CCNE1) and/or cyclin E2 (CCNE2).

E41. The combination of any one of embodiments E23 to E40, wherein the combination is as synergistic combination.

E42. The combination of any one of embodiments E24 to E30, wherein the cancer is HR+/HER2- breast cancer and the additional anti-cancer agent is an endocrine therapeutic agent selected from the group consisting of an aromatase inhibitor, a SERM and a SERD.

E43. The combination of embodiment E42, wherein the endocrine therapeutic agent is letrozole.

E44. The combination of embodiment E42, wherein the endocrine therapeutic agent is fulvestrant.

E45. A combination for use in treating cancer comprising:
(a) a compound of Formula (I):

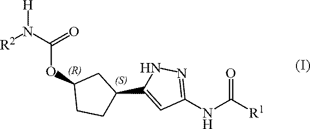

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor.

E46. The combination of embodiment E45, further comprising (c) an additional anti-cancer agent.

E47. The combination of embodiment E45 or E46, wherein the compound of Formula (I) is selected from the group consisting of:
(1R,3S)-3-[3-({[3-(methoxymethyl)-1-methyl-1H-pyrazol-5-yl]carbonyl}amino)-1H-pyrazol-5-yl]cyclopentyl propan-2-ylcarbamate, having the structure:

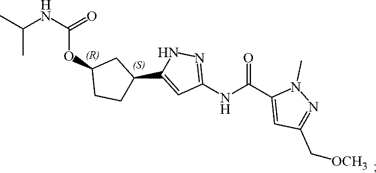

(1R,3S)-3-[3-({[2-(methylsulfonyl)phenyl]acetyl}amino)-1H-pyrazol-5-yl]cyclopentyl (2S)-butan-2-ylcarbamate, having the structure:

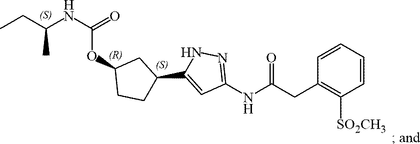

(1R,3S)-3-(3-{[(2-methoxypyridin-4-yl)acetyl]amino}-1H-pyrazol-5-yl)cyclopentyl propylcarbamate, having the structure:

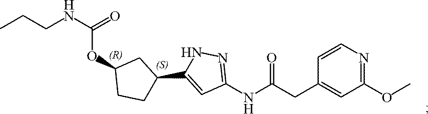

or a pharmaceutically acceptable salt thereof.

E48. The combination of any one of embodiments E45 to E47, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate, having the structure:

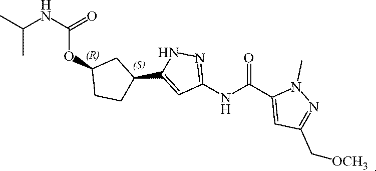

E49. The combination of any one of embodiments E45 to E48, wherein the CDK4/6 inhibitor is palbociclib, or a pharmaceutically acceptable salt thereof.

E50. The combination of any one of embodiments E45 to E49, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E51. The combination of embodiment E50, wherein the cancer is breast cancer.

E52. The combination of embodiment E51, wherein the breast cancer is hormone receptor positive (HR+), human epidermal growth factor receptor 2 negative (HER2-) breast cancer.

E53. The combination of embodiment E51, wherein the breast cancer is triple negative breast cancer (TNBC).

E54. The combination of embodiment E50, wherein the cancer is lung cancer.

E55. The combination of embodiment E54, wherein the lung cancer is small cell lung cancer (SCLC).

E56. The combination of embodiment E54, wherein the lung cancer is non-small cell lung cancer (NSCLC).

E57. The combination of embodiment E50, wherein the cancer is ovarian cancer, peritoneal cancer, or fallopian tube cancer.

E58. The combination of embodiment E57, wherein the cancer is epithelial ovarian cancer (EOC), primary peritoneal carcinomatosis (PPC), or fallopian tube cancer (FTC).

R59. The combination of embodiment E57 or E58, wherein the ovarian cancer is persistent, refractory or recurrent ovarian cancer.

E60. The combination of any one of embodiments E57 to E59, wherein the ovarian cancer is platinum resistant ovarian cancer.

E61. The combination of any one of embodiments E45 to E60, wherein the cancer is advanced or metastatic cancer.

E62. The combination of any one of embodiments E45 to E61, wherein the cancer is characterized by amplification or overexpression of cyclin E1 (CCNE1) and/or cyclin E2 (CCNE2).

E63. The combination of any one of embodiments E45 to E62, wherein the combination is as synergistic combination.

E64. The combination of any one of embodiments E46 to E52, wherein the cancer is HR+/HER2- breast cancer and the additional anti-cancer agent is an endocrine therapeutic agent selected from the group consisting of an aromatase inhibitor, a SERM and a SERD.

E65. The combination of embodiment E64, wherein the endocrine therapeutic agent is letrozole.

E66. The combination of embodiment E64, wherein the endocrine therapeutic agent is fulvestrant.

E67. Use of a combination comprising:
(a) a compound of Formula (I):

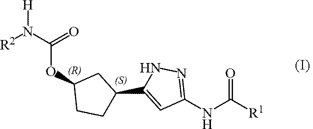

or a pharmaceutically acceptable salt thereof, wherein:
   R¹ is —L—(5-6 membered heteroaryl) or —L—(phenyl), where said 5-6 membered heteroaryl or phenyl is optionally substituted by one to three R³;
   R² is C₁-C₆ alkyl or C₃-C₇ cycloalkyl, where said C₃-C₇ cycloalkyl is optionally substituted by C₁-C₄ alkyl;
   L is a bond or methylene; and
   each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor;
   wherein use of the combination of (a) and (b) is effective in treating cancer.

E68. The use of embodiment E67, wherein the combination further comprises (c) an additional anticancer agent; wherein the use of the combination of (a), (b) and (c) is effective in treating cancer.

E69. The use of embodiment E67 or E68, wherein the compound of Formula (I) is selected from the group consisting of:
(1R,3S)-3-[3-({[3-(methoxymethyl)-1-methyl-1H-pyrazol-5-yl]carbonyl}amino)-1H-pyrazol-5-yl]cyclopentyl propan-2-ylcarbamate, having the structure:

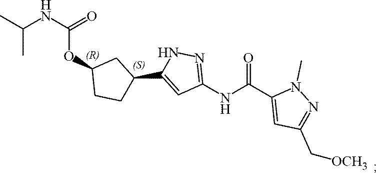

(1R,3S)-3-[3-({[2-(methylsulfonyl)phenyl]acetyl}amino)-1H-pyrazol-5-yl]cyclopentyl (2S)-butan-2-ylcarbamate, having the structure:

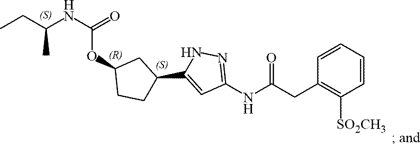

(1R,3S)-3-(3-{[(2-methoxypyridin-4-yl)acetyl]amino}-1H-pyrazol-5-yl)cyclopentyl propylcarbamate, having the structure:

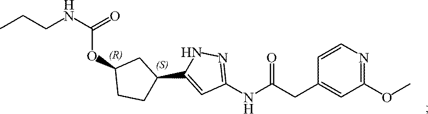

or a pharmaceutically acceptable salt thereof.

E70. The use of any one of embodiments E67 to E69, wherein the compound of Formula (I) is (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate, having the structure:

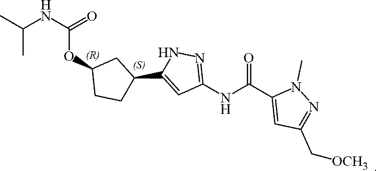

E71. The use of any one of embodiments E67 to E70, wherein the CDK4/6 inhibitor is palbociclib, or a pharmaceutically acceptable salt thereof.

E72. The use of any one of embodiments E67 to E71, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E73. The use of embodiment E72, wherein the cancer is breast cancer.

E74. The use of embodiment E73, wherein the breast cancer is hormone receptor positive (HR+), human epidermal growth factor receptor 2 negative (HER2-) breast cancer.

E75. The use of embodiment E73, wherein the breast cancer is triple negative breast cancer (TNBC).

E76. The use of embodiment E72, wherein the cancer is lung cancer.

E77. The use of embodiment E76, wherein the lung cancer is small cell lung cancer (SCLC).

E78. The use of embodiment E76, wherein the lung cancer is non-small cell lung cancer (NSCLC).

E79. The use of embodiment E72, wherein the cancer is ovarian cancer, peritoneal cancer, or fallopian tube cancer.

E80. The use of embodiment E79, wherein the cancer is epithelial ovarian cancer (EOC), primary peritoneal carcinomatosis (PPC), or fallopian tube cancer (FTC).

E81. The use of embodiment E79 or E80, wherein the ovarian cancer is persistent, refractory or recurrent ovarian cancer.

E82. The use of any one of embodiments E79 to E81, wherein the ovarian cancer is platinum resistant ovarian cancer.

E83. The use of any one of embodiments E67 to E82, wherein the cancer is advanced or metastatic cancer.

E84. The use of any one of embodiments E67 to E83, wherein the cancer is characterized by amplification or overexpression of cyclin E1 (CCNE1) and/or cyclin E2 (CCNE2).

E85. The use of any one of embodiments E67 to E84, wherein the combination is as synergistic combination.

E86. The use of any one of embodiments E68 to E74, wherein the cancer is HR+/HER2- breast cancer and the additional anti-cancer agent is an endocrine therapeutic agent selected from the group consisting of an aromatase inhibitor, a SERM and a SERD.

E87. The use of embodiment E86, wherein the endocrine therapeutic agent is letrozole.

E88. The use of embodiment E86, wherein the endocrine therapeutic agent is fulvestrant.

E89. A method of treating cancer in a subject in need thereof comprising administering to the subject a therapeutically effective amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate.

E90. A method of treating cancer in a subject in need thereof comprising administering to the subject a therapeutically effective amount of a pharmaceutical composition comprising (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate and a pharmaceutically acceptable excipient.

E91. The method of embodiment E89 or E90, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E92. A compound (1R,3S)-3-[3-({[3-(methoxymethyl)-1-methyl-1H-pyrazol-5-yl]carbonyl}-amino)-1H-pyrazol-5-yl]cyclopentyl propan-2-ylcarbamate for use in treating cancer.

E93. A pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate and a pharmaceutically acceptable excipient for use in treating cancer.

E94. The compound of embodiment E92 or pharmaceutical composition of embodiment E93, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

E95. Use of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate for treating cancer.

E96. Use of a pharmaceutical composition comprising (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate and a pharmaceutically acceptable excipient for treating cancer.

E97. The use of embodiment E95 or E96, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

These and other aspects of the invention, including the exemplary specific embodiments listed below, will be apparent from the teachings contained herein.

### EXAMPLES

### Example 1 - Preparation of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A)

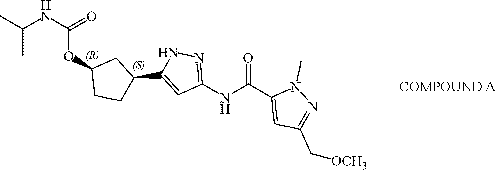 [SMILES: COCc1cc(C(=O)Nc2cc([C@H]3CC[C@@H](OC(=O)NC(C)C)C3)[nH]n2)n(C)n1]

COMPOUND A was prepared as described in Example 13 of U.S. Pat. No. 11,014,911.

### Preparation of Intermediate 1: benzyl {1-tert-butyl-3-[(1S,3R)-3-hydroxycyclopentyl]-1H-pyrazol-5-yl}carbamate; and Intermediate 2: benzyl {1-tert-butvl-3-[(1R,3S)-3-hydroxycyclopentyl]-1H-pyrazol-5-yl}carbamate.

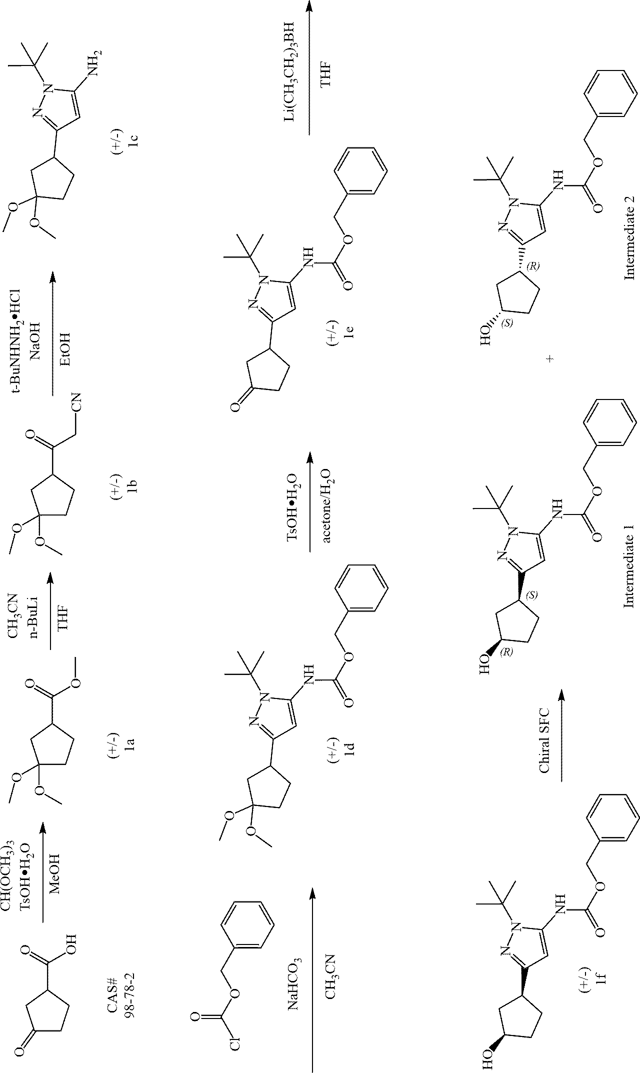

Two parallel reactions, each containing a solution of (±)-3-oxocyclopentanecarboxylic acid (CAS#98-78-2, 900 g, 7.02 mol) in methanol (5 L) at 13° C. were each treated with trimethyl orthoformate (4.47 kg, 42.15 mol, 4.62 L) and 4-toluenesulfonic acid monohydrate (26.72 g, 140.5 mmol). The mixtures were stirred at 13° C. for 25 hours. Each batch was quenched separately with sat. aq NaHCO₃ (1 L), then the two batches were combined and concentrated under vacuum to remove most of the methanol. The residue was diluted with ethyl acetate (4 L), and the layers separated. The aqueous layer was further extracted with ethyl acetate (2 × 1 L). The combined organic layers were washed with sat. aq NaCl (3 × 1 L), dried over magnesium sulfate, filtered, and concentrated under vacuum to give (±)-methyl 3,3-dimethoxycyclopentanecarboxylate (1a, 2.5 kg, 13.28 mol, 94%) as a light yellow oil. ¹H NMR (400 MHz, CHLOROFORM-d) δ = 3.67 (s, 3H), 3.20 (s, 3H), 3.19 (s, 3H), 2.94-2.82 (m, 1H), 2.16-2.00 (m, 2H), 1.99-1.76 (m, 4H).

A solution of n-butyllithium (3.44 L of a 2.5 M solution in hexanes, 8.6 mol) was added to a reactor containing THF (3 L) at -65° C. Anhydrous acetonitrile (453 mL, 353 g, 8.61 mol) was added dropwise, slowly enough to maintain the internal temperature below -55° C. The mixture was stirred for an additional 1 hour at -65° C. A solution of (±)-methyl 3,3-dimethoxycyclopentanecarboxylate (1a, 810 g, 4.30 mol) in THF (1 L) was then added dropwise, slowly enough to maintain the internal temperature below -50° C. After stirring for an additional hour at -65° C., the reaction was quenched with water (4 L), neutralized with aq HCl (1 M) to pH 7, and extracted with ethyl acetate (3 × 3 L). The combined organic layers were washed with sat. aq NaCl (2 × 3 L), dried over magnesium sulfate, filtered, and concentrated under vacuum to give crude (±)-3-(3,3-dimethoxycyclopentyl)-3-oxopropanenitrile (1b, 722 g, 3.66 mol, 85%) as a red oil, which was used without further purification.

Solid sodium hydroxide (131.4 g, 3.29 mol total) was added in portions to a suspension of tert-butylhydrazine hydrochloride (409.4 g, 3.29 mol) in ethanol (3 L) at 16-25° C. Stirring was continued at 25° C. for 1 hour. A solution of crude (±)-3-(3,3-dimethoxycyclopentyl)-3-oxopropanenitrile (1b, 540 g, 2.74 mol) in ethanol was added at 25° C., then the mixture was heated to 75° C. internal and stirred for 30 hours. The reaction was filtered, and the filtrate concentrated under vacuum to give crude product as a red oil. This product was combined with crude from three more identically-prepared batches (each starting with 540 g 1b; 2.16 kg, 10.96 mol total for the 4 batches), and purified by silica gel chromatography (eluting with 0-35% ethyl acetate in petroleum ether), affording (±)-1-tert-butyl-3-(3,3-dimethoxycyclopentyl)-1H-pyrazol-5-amine (1c, 1.60 kg, 5.98 mol, 54% yield) as a red oil. ¹H NMR (CHLOROFORM-d) δ = 5.41 (s, 1H), 3.50 (br. s., 2H), 3.22 (s, 3H), 3.20 (s, 3H), 3.13 (tt, J=7.9, 9.6 Hz, 1H), 2.25 (dd, J=8.0, 13.3 Hz, 1H), 2.09-2.00 (m, 1H), 1.99-1.91 (m, 1H), 1.83 (dd, J=10.8, 12.8 Hz, 2H), 1.78-1.68 (m, 1H), 1.60 (s, 9H).

Benzyl chloroformate (563.6 mL, 676.3 g, 3.96 mol) was added to a chilled (0-5° C.) solution of (±)-1-tert-butyl-3-(3,3-dimethoxycyclopentyl)-1H-pyrazol-5-amine (1c, 530 g, 1.98 mol) in acetonitrile (3.5 L). The mixture was stirred at 23° C. for 2 hours, and then solid sodium hydrogen carbonate (532.9 g, 6.34 mol) was added in portions. Stirring was continued at 23° C. for 26 hours. The resulting suspension was filtered and the filtrate concentrated under vacuum to give crude (±)-benzyl [1-tert-butyl-3-(3,3-dimethoxycyclopentyl)-1H-pyrazol-5-yl]carbamate (1d, 980 g, 1.98 mol max) as a red oil, which was used in the next step without further purification.

A solution of the crude (±)-benzyl [1-tert-butyl-3-(3,3-dimethoxycyclopentyl)-1H-pyrazol-5-yl]carbamate (1d, 980 g, 1.98 mol max) in acetone (2 L) and water (2 L) at 18° C. was treated with 4-toluenesulfonic acid monohydrate (48.75 g, 256.3 mmol). The mixture was heated to 60° C. internal for 20 hours. After concentration under vacuum to remove most of the acetone, the aqueous residue was extracted with dichloromethane (3 × 3 L). The combined organic extracts were dried over sodium sulfate, filtered, and concentrated under vacuum to a crude red oil. This crude product was combined with crude from two other identically-prepared batches (each derived from 1.98 mol 1c, 5.94 mol total for the 3 batches), and purified by silica gel chromatography (eluting with 0-50% ethyl acetate in petroleum ether) to give (±)-benzyl [1-tert-butyl-3-(3-oxocyclopentyl)-1H-pyrazol-5-yl]carbamate (1e, 1.6 kg) as a yellow solid. This solid was stirred in 10:1 petroleum ether/ethyl acetate (1.5 L) at 20° C. for 18 hours. The resulting suspension was filtered, the filter cake washed with petroleum ether (2 × 500 mL), and the solids dried under vacuum to give (±)-benzyl [1-tert-butyl-3-(3-oxocyclopentyl)-1H-pyrazol-5-yl]carbamate (1e, 1.4 kg, 3.9 mol, 66% combined for the three batches). ¹H NMR (DMSO--d₆) δ = 9.12 (br. s., 1H), 7.56-7.13 (m, 5H), 6.03 (s, 1H), 5.12 (s, 2H), 3.41-3.27 (m, 1H), 2.48-2.39 (m, 1H), 2.34-2.10 (m, 4H), 1.98-1.81 (m, 1H), 1.48 (s, 9H).

A solution of (±)-benzyl [1-tert-butyl-3-(3-oxocyclopentyl)-1H-pyrazol-5-yl]carbamate (1e, 320 g, 0.900 mol) in THF (1.5 L) was degassed under vacuum and purged with dry nitrogen (3 cycles), then cooled to -65° C. internal. A solution of lithium triethylborohydride (1.0 M in THF, 1.80 L, 1.80 mol) was added dropwise at a rate which maintained the internal temperature below -55° C., then stirring was continued at -65° C. for 1.5 hours. The reaction mixture was quenched with sat. aq NaHCO₃ (1.5 L) at -40 to -30° C. Hydrogen peroxide (30% aqueous, 700 g) was added to the mixture dropwise, while the internal temperature was maintained at -10 to 0° C. The mixture was stirred at 10° C. for 1 hour, then extracted with ethyl acetate (3 × 2 L). The combined organic layers were washed with sat. aq Na₂SO₃ (2 × 1 L) and sat. aq NaCl (2 × 1 L). The organics were dried over magnesium sulfate, filtered, and concentrated under vacuum to a crude yellow oil. The crude product from this batch was combined with crude from three other, identically-prepared batches (each starting from 0.900 mol 1e, for a total of 3.60 mol) for purification. Before chromatography, the combined mixture showed ~3.3:1 cis/trans ratio by NMR. The combined crude product was purified twice by silica gel chromatography, eluting with 0-50% ethyl acetate in dichloromethane), affording (±)-trans-benzyl [1-tert-butyl-3-(3-hydroxycyclopentyl)-1H-pyrazol-5-yl]carbamate (1f, 960 g) as a light yellow solid, which was further purified by trituration, as described below.

A previous batch of 1f had been obtained from smaller-scale reactions, starting from a total of 120 g 1e (0.34 mol). The columned product from this batch was combined with the columned product from the batch above (which had been derived from 3.60 mol 1e, for a total of 3.94 mol 1e used for all the combined batches), suspended in 10:1 dichloromethane/methanol (1.5 L), and stirred at 20° C. for 16 hours. The suspension was filtered, and the filter cake washed with petroleum ether (2 × 500 mL). The solids were dried under vacuum to give clean (±)-trans-benzyl [1-tert-butyl-3-(3-hydroxycyclopentyl)-1H-pyrazol-5-yl]carbamate (1f, 840 g, 2.35 mol, 60% total yield for all the combined batches) as a white solid. ¹H NMR (400 MHz, DMSO-d₆) δ = 9.07 (br. s., 1H), 7.45-7.27 (m, 5H), 5.92 (s, 1H), 5.11 (s, 2H), 4.57 (d, J=4.5 Hz, 1H), 4.21-4.07 (m, 1H), 2.88 (quin, J=8.6 Hz, 1H), 2.24-2.13 (m, 1H), 1.92-1.78 (m, 1H), 1.78-1.62 (m, 2H), 1.61-1.53 (m, 1H), 1.47 (s, 9H), 1.52-1.43 (m, 1H). MS: 358 [M+H]⁺.

The enantiomers of (±)-trans-benzyl [1-tert-butyl-3-(3-hydroxycyclopentyl)-1H-pyrazol-5-yl]carbamate (1f, 700 g, 1.96 mol) were separated by chiral SFC.

The product from the first-eluting enantiomer peak (310 g solid) was suspended in methanol/petroleum ether (1:10, 1 L) and stirred at 25° C. for 1 hour. The suspension was filtered, the filter pad washed with petroleum ether (2 × 500 mL), and the solids dried under vacuum to give benzyl {1-tert-butyl-3-[(1S,3R)-3-hydroxycyclopentyl]-1H-pyrazol-5-yl}carbamate (Intermediate 1, 255 g, 713 mmol, 36%, >99% ee) as a white solid. ¹H NMR (400 MHz, DMSO-d₆) δ = 9.08 (br. s., 1H), 7.58-7.20 (m, 5H), 5.92 (s, 1H), 5.11 (s, 2H), 4.57 (d, J=4.4 Hz, 1H), 4.19-4.09 (m, 1H), 2.88 (quin, J=8.6 Hz, 1H), 2.24-2.13 (m, 1H), 1.91-1.79 (m, 1H), 1.79-1.61 (m, 2H), 1.61-1.53 (m, 1H), 1.47 (s, 9H), 1.52-1.44 (m, 1H). MS: 358 [M+H]⁺. Optical rotation [α]_{D} +3.76 (c 1.0, MeOH). Chiral purity: >99% ee, retention time 3.371 min. Chiral SFC analysis was performed on a ChiralPak AD-3 150 × 4.6 mm ID, 3 µm column heated to 40° C., eluted with a mobile phase of CO₂ and a gradient of 0-40% methanol+0.05%DEA over 5.5 min, then held at 40% for 3 min; flowing at 2.5 mL/min.

The product from the second-eluting enantiomer peak (300 g solid) was suspended in methanol/petroleum ether (1:10, 1 L) and stirred at 25° C. for 1 hour. The suspension was filtered, the filter pad washed with petroleum ether (2 × 500 mL), and the solids dried under vacuum to give benzyl {1-tert-butyl-3-[(1R,3S)-3-hydroxycyclopentyl]-1H-pyrazol-5-yl}carbamate (Intermediate 2, 255 g, 713 mmol, 36%, 94% ee) as a white solid. ¹H NMR (400 MHz, DMSO-d₆) δ = 9.08 (br. s., 1H), 7.55-7.19 (m, 5H), 5.92 (s, 1H), 5.11 (s, 2H), 4.57 (d, J=4.4 Hz, 1H), 4.23-4.07 (m, 1H), 2.88 (quin, J=8.7 Hz, 1H), 2.23-2.14 (m, 1H), 1.90-1.79 (m, 1H), 1.77-1.61 (m, 2H), 1.61-1.53 (m, 1H), 1.47 (s, 9H), 1.52-1.44 (m, 1H). MS: 358 [M+H]⁺. Optical rotation [α]o -2.43 (c 1.0, MeOH). Chiral purity: 94% ee, retention time 3.608 min. Chiral SFC analysis was performed on a ChiralPak AD-3 150 × 4.6 mm ID, 3 µm column heated to 40° C., eluted with a mobile phase of CO₂ and a gradient of 0-40% methanol+0.05%DEA over 5.5 min, then held at 40% for 3 min; flowing at 2.5 mL/min.

A sample of the second-eluting enantiomer from a previous batch with [α]_{D} -3.1 (c 1.1, MeOH) and 96% ee was crystalized from dichloroethane/pentane. A crystal structure was obtained by small-molecule X-ray crystallography, which showed (1R,3S) geometry. The absolute stereochemistry of Intermediate 2 was thus assigned (1R,3S) based on its comparable optical rotation and order of elution in the analytical method. Intermediate 1, the enantiomer of Intermediate 2, was thus assigned (1S,3R) stereochemistry.

### Preparation of Intermediate 11B: (1-tert-butyl-3-[(1S,3R)-3-{[tert-butyl(dimethyl)-silyl]oxy}cyclopentyl]-1H-pyrazol-5-amine

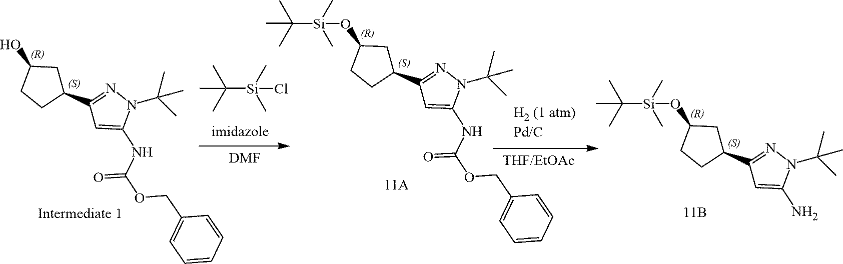

Benzyl {1-tert-butyl-3-[(1S,3R)-3-hydroxycyclopentyl]-1H-pyrazol-5-yl}carbamate (Intermediate 1, 20 g, 56 mmol) and imidazole (5.71 g, 83.9 mmol) were dissolved in DMF (200 mL) with sonication. While the solution was at room temperature, tertbutyldimethylsilyl chloride (11.0 g, 72.7 mmol) was added in portions. After the addition was complete, the clear solution was stirred at 25° C. for 1 hour. The solvents were removed under vacuum and the residue partitioned between ethyl acetate (500 mL) and sat. aq NaCl (200 mL). The organic layer was dried over sodium sulfate, filtered, and concentrated to give crude benzyl {1-tert-butyl-3-[(1S,3R)-3-{[tert-butyl(dimethyl)silyl]-oxy}cyclopentyl]-1H-pyrazol-5-yl}carbamate (11A, 26 g, 99%) as a colorless oil. MS: 472 [M+H]⁺.

Crude benzyl {1-tert-butyl-3-[(1S,3R)-3-{[tert-butyl(dimethyl)silyl]oxy}cyclopentyl]-1H-pyrazol-5-yl}carbamate (11A, 26 g, 55 mmol) was dissolved in ethyl acetate (100 mL) and THF (100 mL). Added Pd/C (50% wet, 4 g), degassed the solution, and stirred at 25° C. under a hydrogen balloon for 2 hours. The mixture was then filtered, and the filtrate concentrated under vacuum to give crude 1-tert-butyl-3-[(1S,3R)-3-{[tert-butyl(dimethyl)silyl]oxy}cyclopentyl]-1H-pyrazol-5-amine (11B, 19 g, >99%) as a light yellow oil. MS: 338 [M+H]⁺.

### Preparation of Intermediate 5: Lithium 3-(methoxymethyl)-1-methyl-1H-pyrazole-5-carboxylate.

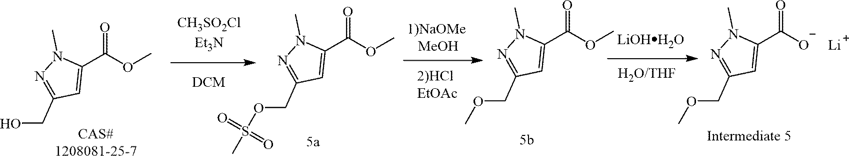

A solution of methanesulfonyl chloride (11.32 g, 98.8 mmol) in dichloromethane (50 mL) was added dropwise to a cooled (0° C.) mixture of methyl 3-(hydroxymethyl)-1-methyl-1H-pyrazole-5-carboxylate (CAS# 1208081-25-7, 15.0 g, 88.1 mmol) and diisopropylethyl amine (14.8 g, 115 mmol) in dichloromethane (250 mL). The mixture was stirred at 0° C. for 45 minutes after the addition was complete. The reaction mixture was washed with sat. aq NH₄Cl, and the organic layer dried over sodium sulfate, filtered, and concentrated to give methyl 1-methyl-3-{[(methylsulfonyl)oxy]methyl}-1H-pyrazole-5-carboxylate (5a, 22.6 g, >99%) as a yellow oil, which was used without further purification. ¹H NMR (400 MHz, CHLOROFORM-d) δ = 6.98 (s, 1H), 5.26 (s, 2H), 4.20 (s, 3H), 3.91 (s, 3H), 3.03 (s, 3H).

A solution of methyl 1-methyl-3-{[(methylsulfonyl)oxy]methyl}-1H-pyrazole-5-carboxylate (5a, 22.6 g, 91.0 mmol) in methanol (200 mL) at room temperature was treated with solid sodium methoxide (9.84 g, 182 mmol) in small portions. The reaction was heated to 70° C. for 30 minutes. TLC suggested partial hydrolysis of the ester, so to re-esterify, the cloudy mixture was acidified with 4 M HCl in ethyl acetate (40 mL, 160 mmol), and heating continued at 70° C. for 5 hours. The mixture was concentrated to dryness, leaving a white solid. This solid was extracted with ethyl acetate/petroleum ether (⅓, 3 × 200 mL). The combined extracts were concentrated to dryness, then the residual solid re-extracted with ethyl acetate/petroleum ether (⅓, 100 mL), dried over sodium sulfate, filtered, and concentrated to give methyl 3-(methoxymethyl)-1-methyl-1H-pyrazole-5-carboxylate (5b, 14.5 g, 86%, 80% pure by NMR) as a light yellow liquid which solidified on standing. Major component only: ¹H NMR (400 MHz, CHLOROFORM-d) δ = 6.83 (s, 1H), 4.45 (s, 2H), 4.16 (s, 3H), 3.88 (s, 3H), 3.39 (s, 3H).

A solution of methyl 3-(methoxymethyl)-1-methyl-1H-pyrazole-5-carboxylate (5b, 14.5 g, 78.7 mmol) and lithium hydroxide monohydrate (3.47 g, 82.7 mmol) in THF (150 mL) and water (50 mL) was stirred at room temperature for 16 hours. The THF was removed under vacuum, and the residue dissolved in water (100 mL) and extracted with dichloromethane (3 × 30 mL). The organic layers were discarded. The aqueous layer was concentrated and dried under vacuum to give lithium 3-(methoxymethyl)-1-methyl-1H-pyrazole-5-carboxylate (Intermediate 5, 12.85 g, 92%) as a yellow solid. ¹H NMR (400 MHz, DMSO-d₆) δ = 6.37 (s, 1H), 4.24 (s, 2H), 4.01 (s, 3H), 3.20 (s, 3H). MS: 171 [M+H]⁺.

### Preparation of Compound A:

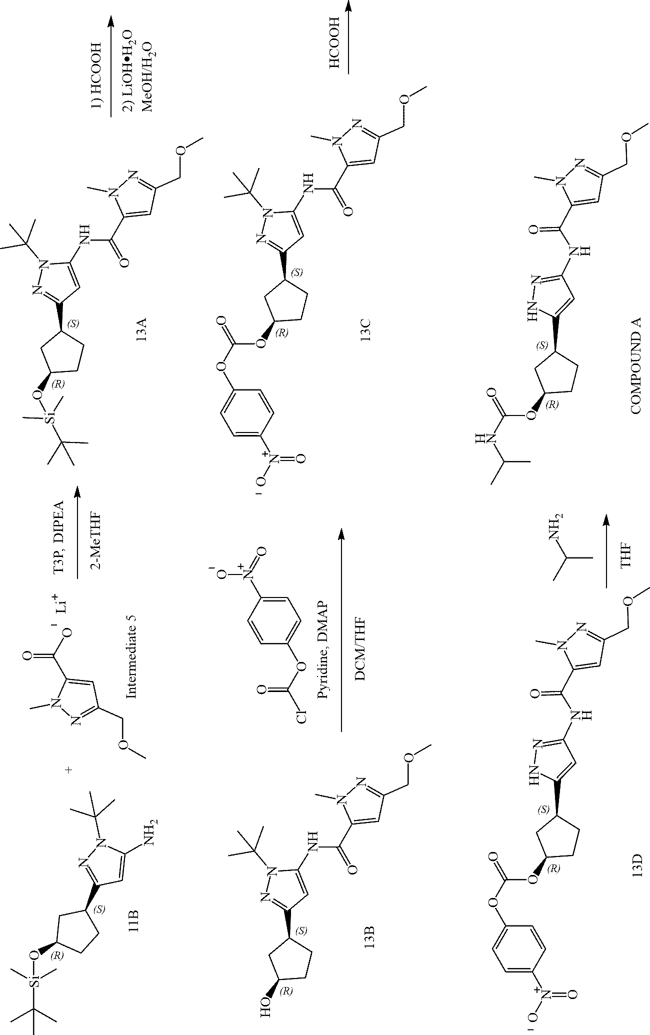

Propylphosphonic anhydride (T3P®, 50 wt% solution in EtOAc, 50.3 g, 79.1 mmol) was added to a room temperature (26° C.) solution of 1-tert-butyl-3-[(1S,3R)-3-{[tert-butyl(dimethyl)silyl]oxy}cyclopentyl]-1H-pyrazol-5-amine (11B, 8.90 g, 26.4 mmol), lithium 3-(methoxymethyl)-1-methyl-1H-pyrazole-5-carboxylate (Intermediate 5, 5.83 g, 34.3 mmol), and diisopropylethyl amine (10.2 g, 79.1 mmol) in 2-methyltetrahydrofuran (100.0 mL). The resulting mixture was stirred at this temperature for 18 hours. After concentrating the mixture to dryness, the residue was dissolved in dichloromethane (150 mL), and the solution washed sequentially with water (2 × 30 mL), sat. aq NaHCO₃ (2 × 30 mL) and sat. aq NaCl (30 mL). The organic layer was dried over sodium sulfate, filtered, and concentrated to give crude N-{1-tert-butyl-3-[(1S,3R)-3-{[tert-butyl(dimethyl)silyl]oxy}cyclopentyl]-1H-pyrazol-5-yl}-3-(methoxymethyl)-1-methyl-1 H-pyrazole-5-carboxamide (13A, 12.9 g, 100%) as an oil. MS: 490 [M+H]⁺.

The crude N-{1-tert-butyl-3-[(1S,3R)-3-{[tert-butyl(dimethyl)silyl]oxy}cyclopentyl]-1H-pyrazol-5-yl}-3-(methoxymethyl)-1-methyl-1H-pyrazole-5-carboxamide (13A, 12.9 g, 26.3 mmol) was dissolved in formic acid (80 mL) and stirred at room temperature (27° C.) for 30 minutes. The mixture was concentrated to dryness, and the residue dissolved in methanol (80 mL). A solution of lithium hydroxide monohydrate (3.43 g, 81.8 mmol) in water (15 mL) was added, and the mixture stirred at room temperature (27° C.) for 1 hour. The mixture was concentrated to dryness, and the residue was purified by silica gel chromatography (eluting with 0-80% ethyl acetate in petroleum ether) to give N-{1-tert-butyl-3-[(1S,3R)-3-hydroxycyclopentyl]-1H-pyrazol-5-yl}-3-(methoxymethyl)-1-methyl-1H-pyrazole-5-carboxamide (13B, 8.0 g, 78%) as a yellow gum. MS: 376 [M+H]⁺.

A solution of N-{1-tert-butyl-3-[(1S,3R)-3-hydroxycyclopentyl]-1H-pyrazol-5-yl}-3-(methoxymethyl)-1-methyl-1H-pyrazole-5-carboxamide (13B, 8.0 g, 21 mmol) in dichloromethane (80 mL) and THF (80 mL) was treated with DMAP (260 mg, 2.13 mmol), pyridine (5.06 g, 63.9 mmol), and 4-nitrophenyl chloroformate (8.59 g, 42.6 mmol). The resulting yellow suspension was stirred at room temperature for 18 hours. The reaction mixture was concentrated to dryness and purified by silica gel chromatography (eluting with 0-45% ethyl acetate in petroleum ether) to give (1R,3S)-3-[1-tert-butyl-5-({[3-(methoxymethyl)-1-methyl-1H-pyrazol-5-yl]carbonyl}amino)-1H-pyrazol-3-yl]cyclopentyl 4-nitrophenyl carbonate (13C, 10.6 g, 92%) as a light brown gum. MS: 541 [M+H]⁺.

A solution of (1R,3S)-3-[1-tert-butyl-5-({[3-(methoxymethyl)-1-methyl-1H-pyrazol-5-yl]carbonyl}amino)-1H-pyrazol-3-yl]cyclopentyl 4-nitrophenyl carbonate (13C, 10.6 g, 19.6 mmol) in formic acid (80 mL) was stirred at 70° C. for 18 hours. The solution was concentrated to dryness. The residue was dissolved in dichloromethane (150 mL) and the solution neutralized with sat. aq NaHCO₃. The organic layer was washed with water (30 mL) and sat. aq NaCl (30 mL), dried over sodium carbonate, filtered, and concentrated to give crude (1R,3S)-3-[3-({[3-(methoxymethyl)-1-methyl-1H-pyrazol-5-yl]carbonyl}amino)-1H-pyrazol-5-yl]cyclopentyl 4-nitrophenyl carbonate (13D, 8.5 g, 90%, 86% pure by LCMS) as a light yellow glass. MS: 485 [M+H]⁺. A room temperature (27° C.) solution of crude (1R,3S)-3-[3-({[3-(methoxymethyl)-1-methyl-1H-pyrazol-5-yl]carbonyl}amino)-1H-pyrazol-5-yl]cyclopentyl 4-nitrophenyl carbonate (13D, 1.7 g, 3.5 mmol) and 2-propylamine (1.04 g, 17.5 mmol) in THF (30 mL) was stirred for 6 hours. The solution was concentrated to dryness, and the residue was combined with the residue from a second batch which had been derived from 1.7 g, 3.5 mmol 13D (total 6.27 mmol 13D consumed for the combined two batches) to give 3.2 g crude product. This product was purified by preparative HPLC on a Phenomenex Gemini C18 250*50 mm*10 µm column, eluting with 15-45% water (0.05% ammonium hydroxide v/v) in acetonitrile. After lyophilization, (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate (COMPOUND A, 2.06 g, 78%) was obtained as a white crystalline solid monohydrate. MS: 405 [M+H]⁺. ¹H NMR (400 MHz, DMSO-d6) δ = 12.23 (br s, 1H), 10.73 (br s, 1H), 7.11 (s, 1H), 6.96 (br d, J=7.0 Hz, 1H), 6.41 (br s, 1H), 5.00 (br s, 1H), 4.33 (s, 2H), 4.04 (s, 3H), 3.57 (qd, J=6.6, 13.4 Hz, 1H), 3.26 (s, 3H), 3.17-2.96 (m, 1H), 2.48-2.39 (m, 1H), 2.03 (br d, J=6.8 Hz, 1H), 1.95-1.83 (m, 1H), 1.73 (br d, J=8.5 Hz, 2H), 1.61 (br s, 1H), 1.03 (br d, J=6.3 Hz, 6H). Optical rotation [α]o +4.8 (c 1.0, MeOH). Chiral purity: >99% ee by chiral analytical SFC. Anal. Calcd for C₁₉H₂₈N₆O₄-H₂O: C, 54.02; H, 7.16; N, 19.89. Found: C, 53.94; H, 7.22; N, 19.81.

### Example 2 - Preparation of (1R,3S)-3-[3-({[2-(methylsulfonyl)phenyl]acetyl}amino)-1H-pyrazol-5-yllcyclopentyl (2S)-butan-2-ylcarbamate (COMPOUND B)

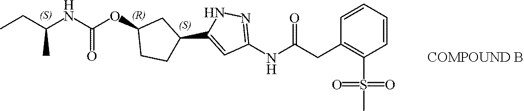 [SMILES: CC[C@H](C)NC(=O)O[C@@H]1CC[C@H](c2cc(NC(=O)Cc3ccccc3S(C)(=O)=O)n[nH]2)C1]

COMPOUND B was prepared as described below and characterized as in Example 370 U.S. Pat.No. 11,014,911.

### Preparation of Intermediate 1A : (1R,3S)-3-(5-{[(benzyloxy)carbonyl]amino}-1-tert-butyl-1H-pyrazol-3-yl)cyclopentyl 4-nitrophenyl carbonate

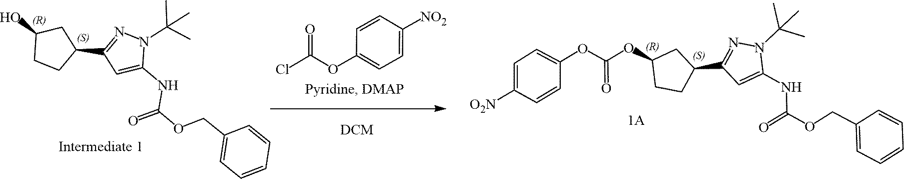

A room-temperature solution of benzyl {1-tert-butyl-3-[(1S,3R)-3-hydroxycyclopentyl]-1H-pyrazol-5-yl}carbamate (Intermediate 1, 5.00 g, 14.0 mmol) and 4-nitrophenyl chloroformate (4.23 g, 21.0 mmol) in anhydrous dichloromethane (50 mL) was treated with pyridine (3.40 mL, 42.0 mmol) and 4-(dimethylamino)pyridine (170 mg, 1.4 mmol). After stirring at room temperature overnight, the solution was concentrated and purified by silica gel chromatography (eluting with 0-100% ethyl acetate in n-heptane) to give (1R,3S)-3-(5-{[(benzyloxy)carbonyl]amino}-1-tert-butyl-1H-pyrazol-3-yl)cyclopentyl 4-nitrophenyl carbonate (1A, 7.30 g, 100%) as a solid foam. ¹H NMR (400 MHz, CHLOROFORM-d) δ = 8.24-8.14 (m, 2H), 7.36-7.22 (m, 7H), 6.21 (br. s., 1H), 6.06 (br. s., 1H), 5.25 - 5.15 (m, 1H), 5.12 (s, 2H), 3.15-2.97 (m, 1H), 2.58-2.47 (m, 1H), 2.09-1.78 (m, 5H), 1.51 (s, 9H). MS: 523 [M+H]⁺.

### Preparation of Intermediate 4B: (1R,3S)-3-(5-amino-1-tert-butyl-1H-pyrazol-3-yl)cyclopentyl (2S)-butan-2-ylcarbamate

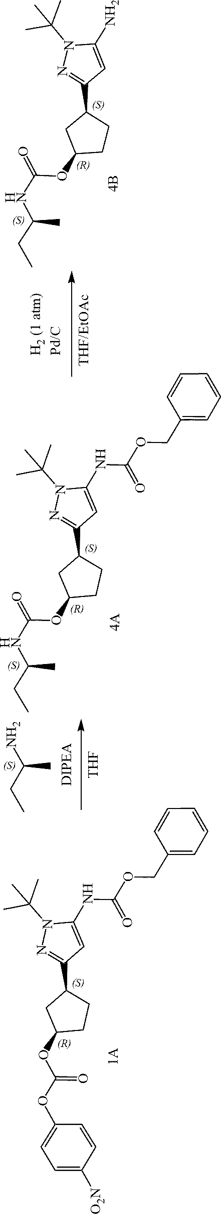

A solution of (1R,3S)-3-(5-{[(benzyloxy)carbonyl]amino}-1-tert-butyl-1H-pyrazol-3-yl)cyclopentyl 4-nitrophenyl carbonate (1A, 22.0 g, 42.1 mmol), (S)-(+)-sec-butylamine (4.00 g, 54.7 mmol), and diisopropylethyl amine (36.7 mL, 211 mmol) in THF (300 mL) was stirred at 10° C. for 16 hours. The mixture was concentrated to dryness, and the residue diluted with ethyl acetate (500 mL). The solution was washed with 1 M aq NaOH (4 × 200 mL) and sat. aq NaCl (100 mL). The organic layer was dried over sodium sulfate, filtered, and concentrated to give crude benzyl {3-[(1S,3R)-3-{[(2S)-butan-2-ylcarbamoyl]oxy}cyclopentyl]-1-tert-butyl-1H-pyrazol-5-yl}carbamate (4A, 18 g, 94%, ~80% pure by LCMS). MS: 479 [M+Na]⁺.

A room temperature (10° C.) solution of the crude benzyl {3-[(1S,3R)-3-{[(2S)-butan-2-ylcarbamoyl]oxy}cyclopentyl]-1-tert-butyl-1H-pyrazol-5-yl}carbamate (4A, 18 g, 39 mmol) in ethyl acetate (200 mL) and THF (100 mL) was degassed and treated with Pd/C catalyst (wet, 5 g). The suspension was stirred under a hydrogen balloon for 16 hours. The mixture was filtered to remove the catalyst, the filtrate was concentrated to dryness. For purification, this batch was combined with a second batch of crude derived by the same method from 20 g 4A (total for both batches: 38 g, 83 mmol) and purified by preparative HPLC on a Phenomenex Gemini C18 250*50 mm*10 µm column, eluting with 30-50% water (0.05% ammonium hydroxide v/v) in acetonitrile. After lyophilization, (1R,3S)-3-(5-amino-1-tert-butyl-1H-pyrazol-3-yl)cyclopentyl (2S)-butan-2-ylcarbamate (4B, 20.1 g, 75% for the combined batches). MS: 323 [M+H]⁺. ¹H NMR (400 MHz, DMSO-d₆) δ = 6.86 (br d, J=8.3 Hz, 1H), 5.22 (s, 1H), 4.94 (br s, 1H), 4.82-4.49 (m, 2H), 3.46-3.36 (m, 1H), 2.90-2.71 (m, 1H), 2.38-2.24 (m, 1H), 1.91-1.75 (m, 2H), 1.74-1.53 (m, 3H), 1.52-1.46 (m, 9H), 1.43-1.27 (m, 2H), 1.01 (d, J=6.5 Hz, 3H), 0.81 (t, J=7.4 Hz, 3H). Optical rotation [α]o +4.0 (c 1.3, MeOH). Chiral purity: 98% de by chiral analytical SFC.

### Preparation of COMPOUND B: (1R,3S)-3-[3-({[2-(methylsulfonyl)phenyl]acetyl}-amino)-1H-pyrazol-5-yl]cyclopentyl (2S)-butan-2-ylcarbamate

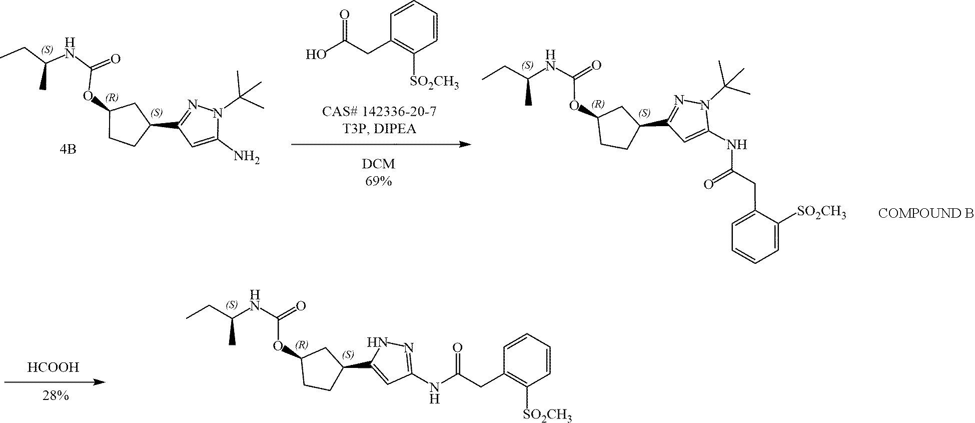

Intermediate 4B was converted to the 2-(methylsulfonyl)phenylacetamide intermediate under the conditions described in Example 3 (below) for conversion of Intermediate 1C to Intermediate 1D. The phenylacetamide intermediate was converted to COMPOUND B under the conditions described in Example 3 for conversion of Intermediate 1D to COMPOUND C.

### Example 3 - Preparation of (1R,3S)-3-(3-{[(2-methoxypyridin-4-yl)acetyl]amino}-1H-pyrazol-5-yl)cyclopentyl propylcarbamate (COMPOUND C)

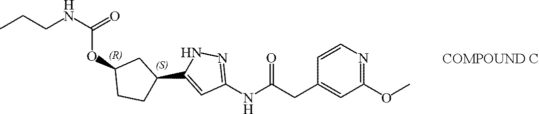 [SMILES: CCCNC(=O)O[C@@H]1CC[C@H](c2cc(NC(=O)Cc3ccnc(OC)c3)n[nH]2)C1]

COMPOUND C was prepared as described in Example 1 of U.S. Pat. No. 11,014,911.

### Preparation of Compound C

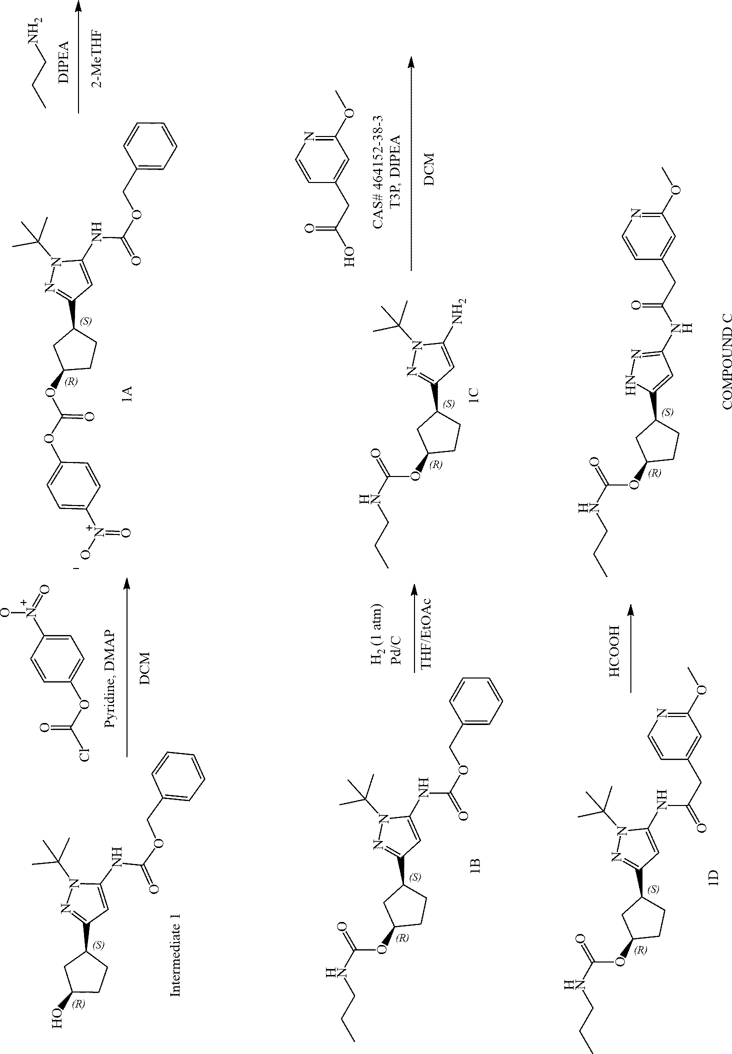

A room-temperature solution of benzyl {1-tert-butyl-3-[(1S,3R)-3-hydroxycyclopentyl]-1H-pyrazol-5-yl}carbamate (Intermediate 1, 5.00 g, 14.0 mmol) and 4-nitrophenyl chloroformate (4.23 g, 21.0 mmol) in anhydrous dichloromethane (50 mL) was treated with pyridine (3.40 mL, 42.0 mmol) and 4-(dimethylamino)pyridine (170 mg, 1.4 mmol). After stirring at room temperature overnight, the solution was concentrated and purified by silica gel chromatography (eluting with 0-100% ethyl acetate in n-heptane) to give (1R,3S)-3-(5-{[(benzyloxy)carbonyl]amino}-1-tert-butyl-1H-pyrazol-3-yl)cyclopentyl 4-nitrophenyl carbonate (1A, 7.30 g, 100%) as a solid foam. ¹H NMR (400 MHz, CHLOROFORM-d) δ = 8.24-8.14 (m, 2H), 7.36-7.22 (m, 7H), 6.21 (br. s., 1H), 6.06 (br. s., 1H), 5.25 -5.15 (m, 1H), 5.12 (s, 2H), 3.15-2.97 (m, 1H), 2.58-2.47 (m, 1H), 2.09-1.78 (m, 5H), 1.51 (s, 9H). MS: 523 [M+H]⁺.

A solution of (1R,3S)-3-(5-{[(benzyloxy)carbonyl]amino}-1-tert-butyl-1H-pyrazol-3-yl)cyclopentyl 4-nitrophenyl carbonate (1A, 36 g, 69 mmol) in 2-methlytetrahydrofuran (300 mL) was cooled to 10° C. Diisopropylethyl amine (26.7 g, 36 mL, 207 mmol) and propan-1-amine (6.11 g, 8.52 mL, 103 mmol) were added, and the solution stirred at 10° C. for 16 hours. After concentrating to dryness, the residue was diluted with ethyl acetate (600 mL), washed with 1 M NaOH (4 × 200 mL), and then with sat. aq NaCl (100 mL). The organic layer was dried over sodium sulfate, filtered, and concentrated to give crude benzyl (1-tert-butyl-3-{(1S,3R)-3-[(propylcarbamoyl)oxy]cyclopentyl}-1H-pyrazol-5-yl)carbamate (1B, 30 g, 98%), which was used without further purification.

A room-temperature (20-25° C.) suspension of Pd/C (50% H₂O, 8 g) and crude benzyl (1-tert-butyl-3-{(1S,3R)-3-[(propylcarbamoyl)oxy]cyclopentyl}-1H-pyrazol-5-yl)carbamate (1B, 30 g, 68 mmol) in ethyl acetate (300 mL) and THF (150 mL) was degassed and purged with hydrogen (3 cycles), then stirred at room temperature under a hydrogen balloon for 16 hours. The suspension was filtered, the filtrate concentrated under vacuum, and the residue crystallized from ethyl acetate (50 mL) and petroleum ether (300 mL), affording (1R,3S)-3-(5-amino-1-tert-butyl-1H-pyrazol-3-yl)cyclopentyl propylcarbamate (1C, 17.65 g, 84%) as a white solid. ¹H NMR (400 MHz, DMSO-d₆) δ = 7.00 (br t, J=5.6 Hz, 1H), 5.23 (s, 1H), 4.95 (br s, 1H), 4.82-4.58 (m, 2H), 2.91 (q, J=6.6 Hz, 2H), 2.85-2.73 (m, 1H), 2.37-2.21 (m, 1H), 1.92-1.76 (m, 2H), 1.72-1.52 (m, 3H), 1.48 (s, 9H), 1.44-1.32 (m, 2H), 0.82 (t, J=7.4 Hz, 3H). MS: 309 [M+H]⁺. ]⁺. Optical rotation [α]_{D} -4.04 (c 0.89, MeOH). Chiral purity: 98% ee by chiral analytical SFC.

A cooled (10° C.) mixture of (1R,3S)-3-(5-amino-1-tert-butyl-1H-pyrazol-3-yl)cyclopentyl propylcarbamate (1C, 8.65 g, 28.05 mmol), (2-methoxypyridin-4-yl)acetic acid (CAS# 464152-38-3, 5.86 g, 33.7 mmol) diisopropylethyl amine (14.7 mL, 84.1 mmol) and propylphosphonic anhydride (T3P®, 50 wt% solution in EtOAc, 53.5 g, 84.1 mmol) in dichloromethane (250 mL) was stirred for 16 hours. The reaction was quenched with sat. aq Na₂CO₃ (20 mL) and extracted with dichloromethane (100 mL). The organic layer was washed with more sat. aq Na₂CO₃ (2 × 200 mL) and sat. aq NaCl (100 mL), dried over anhydrous sodium sulfate, filtered, and concentrated to dryness. For purification, this batch was combined with two other similarly-prepared batches derived from 1.0 g and 8.0 g 1C (total SM for the three batches = 17.65 g, 57.23 mmol 1C). Silica gel chromatography (eluting with 0-60% EtOAc/petroleum ether) gave (1R,3S)-3-(1-tert-butyl-5-{[(2-methoxypyridin-4-yl)acetyl]amino}-1H-pyrazol-3-yl)cyclopentyl propylcarbamate (1D, 25 g, 95% yield for the combined batches). MS: 458 [M+H]⁺.

A solution of (1R,3S)-3-(1-tert-butyl-5-{[(2-methoxypyridin-4-yl)acetyl]amino}-1H-pyrazol-3-yl)cyclopentyl propylcarbamate (1D, 20.5 g, 44.8 mmol) in formic acid (50 mL) was stirred at 75° C. for 20 hours. For purification, this batch was combined with a smaller batch (derived from 4.50 g, 9.84 mmol 1D, for a total of 25.0 g, 54.6 mmol), concentrated to dryness, and purified by preparative HPLC [Phenomenex Gemini C18 250 × 50 mm × 10 µm column; eluting with a gradient of water (0.05% ammonium hydroxide v/v) in ACN over 15 minutes; flowing at 110 mL/min]. Pure (1R,3S)-3-(3-{[(2-methoxypyridin-4-yl)acetyl]amino}-1H-pyrazol-5-yl)cyclopentyl propylcarbamate (COMPOUND C), 16.61 g, 76% yield for the combined batches) as a pale yellow solid. ¹H NMR (400 MHz, CHLOROFORM-d) δ = 11.62-9.81 (m, 1H), 9.06 (br s, 1H), 8.06 (d, J=5.3 Hz, 1H), 6.79 (d, J=5.3 Hz, 1H), 6.66 (s, 1H), 6.50 (s, 1H), 5.24-4.94 (m, 2H), 3.88 (s, 3H), 3.58 (s, 2H), 3.19-2.83 (m, 3H), 2.54-2.28 (m, 1H), 2.04 (br s, 1H), 1.97-1.70 (m, 4H), 1.54-1.34 (m, 2H), 0.85 (br t, J=7.0 Hz, 3H). MS: 402 [M+H]⁺. Optical rotation [a]_{D} +17.1 (c 1.06, MeOH). Chiral purity: 96% ee by chiral analytical SFC.

### Example 4 - Biochemical Assays

### CDK2/Cyclin E1 Full Length Mobility Shift Assay

The purpose of CDK2/Cyclin E1 assay is to evaluate the inhibition (% inhibition, Kᵢₐₚₚ and Kᵢ values) of small molecule inhibitors by using a fluorescence-based microfluidic mobility shift assay. CDK2/Cyclin E1 full length catalyzes the production of ADP from ATP that accompanies the phosphoryl transfer to the substrate peptide FL-Peptide-18 (5-FAM-QSPKKG-CONH₂, CPC Scientific, Sunnyvale, CA) (SEQ ID NO:1). The mobility shift assay electrophoretically separates the fluorescently labeled peptides (substrate and phosphorylated product) following the kinase reaction. Both substrate and product are measured and the ratio of these values is used to generate % conversion of substrate to product by the LabChip EZ Reader. Wild-type CDK2/wild-type full length Cyclin E1 enzyme complex was produced in-house (baculoviral expression, LJIC-2080/LJIC-2103) and phosphorylated by CDK7/Cyclin H1/Mat1 enzyme complex with CDK2:CDK7 ratio of 50:1 (concentration mg/mL) in the presence of 10 mM MgCl₂ and 5 mM ATP at room temperature for one hour. Typical reaction solutions (50 µL final reaction volume) contained 2% DMSO (± inhibitor), 4 mM MgCl₂, 1 mM DTT, 150 µM ATP (ATP Kₘ = 67.4 µM), 0.005% Tween-20, 3 µM FL-Peptide-18, and 0.36 nM (catalytically competent active site) phosphorylated wild-type full length CDK2/Cyclin E1 enzyme complex in 25 mM HEPES buffer at pH 7.15. The assay was initiated with the addition of ATP, following a fifteen minutes pre-incubation of enzyme and inhibitor at room temperature in the reaction mixture. The reaction was stopped after 45 minutes at room temperature by the addition of 50 µL of 80 mM EDTA. The Kᵢ value was determined from the fit of the data to the Morrison tight-binding competitive inhibition equation with the enzyme concentration as a variable^{1,}².

### CDK4/Cyclin D₁ Mobility Shift Assay

The purpose CDK4/Cyclin D₁ assay is to evaluate the inhibition (% inhibition, Kᵢₐₚₚ and Kᵢ values) in the presence of small molecule inhibitors by using a fluorescence based microfluidic mobility shift assay. CDK4/Cyclin D₃ catalyses the production of ADP from ATP that accompanies the phosphoryl transfer to the substrate peptide 5-FAM-Dyrktide (5-FAM-RRRFRPASPLRGPPK) (SEQ ID NO:2). The mobility shift assay electrophoretically separates the fluorescently labelled peptides (substrate and phosphorylated product) following the kinase reaction. Both substrate and product are measured and the ratio of these values is used to generate % conversion of substrate to product by the LabChip EZ Reader. Typical reaction solutions contained 2% DMSO (± inhibitor), 10 mM MgCl₂, 1 mM DTT, 3.5 mM ATP, 0.005% TW-20, 3 µM 5-FAM-Dyrktide, 3 nM (active sites) activated CDK4/Cyclin D₁ in 40 mM HEPES buffer at pH 7.5. Inhibitor Kᵢ determinations for activated CDK4/Cyclin D₁ (2007 E1/2008 +PO4) were initiated with the addition of ATP (50 µL final reaction volume), following an eighteen minute pre-incubation of enzyme and inhibitor at 22° C. in the reaction mix. The reaction was stopped after 195 minutes by the addition of 50 µL of 30 mM EDTA. Kᵢ determinations were made from a plot of the fractional velocity as a function of inhibitor concentration fit to the Morrison equation with the enzyme concentration as a variable.

### CDK6/Cyclin D₃ Mobility Shift Assay

The purpose of the CDK6/Cyclin D₃ assay is to evaluate the inhibition (% inhibition, Kᵢₐₚₚ and Kᵢ values) in the presence of small molecule inhibitors by using a fluorescence based microfluidic mobility shift assay. CDK6/Cyclin D₃ catalyses the production of ADP from ATP that accompanies the phosphoryl transfer to the substrate peptide 5-FAM-Dyrktide (5-FAM-RRRFRPASPLRGPPK) (SEQ ID NO:2). The mobility shift assay electrophoretically separates the fluorescently labelled peptides (substrate and phosphorylated product) following the kinase reaction. Both substrate and product are measured and the ratio of these values is used to generate % conversion of substrate to product by the LabChip EZ Reader. Typical reaction solutions contained 2% DMSO (± inhibitor), 2% glycerol, 10 mM MgCl₂, 1 mM DTT, 3.5 mM ATP, 0.005% Tween 20 (TW-20), 3 µM 5-FAM-Dyrktide, 4 nM (active sites) activated CDK6/Cyclin D₃ in 40 mM HEPES buffer at pH 7.5.

Inhibitor Kᵢ determinations for activated CDK6/Cyclin D₃ (LJIC-2009G1/2010 +PO₄) were initiated with the addition of ATP (50 µL final reaction volume), following an eighteen minute pre-incubation of enzyme and inhibitor at 22° C. in the reaction mix. The reaction was stopped after 95 minutes by the addition of 50 µL of 30 mM EDTA. Kᵢ determinations were made from a plot of the fractional velocity as a function of inhibitor concentration fit to the Morrison equation with the enzyme concentration as a variable. For fitting tight-binding inhibitor data generated by CDK4 and CDK6 mobility shift assays, equations and principles are derived from Morrison, J. F. (1969) Kinetics of the reversible inhibition of enzyme-catalysed reactions by tight-binding inhibitors, Biochimica et biophysica acta 185, 269-286; and Murphy, D. J. (2004) and Determination of accurate Kᵢ values for tight-binding enzyme inhibitors: an in silico study of experimental error and assay design, Analytical biochemistry 327, 61-67.

### Biological Activity Data

Biological activity data (Ki (nM)) for COMPOUNDs A, B, C and D are provided in Table 1 below.

TABLE 1

| Compound | CDK2/cyclin E₁ Ki (nM) | CDK4/cyclin D₁ Ki (nM) | CDK6/cyclin D₃ Ki (nM) |
|---|---|---|---|
| COMPOUND A (Example 1) | 1.16 | 243 | 465 |
| COMPOUND B (Example 2) | 0.28 | 107 | 254 |
| COMPOUND C (Example 3) | 0.55 | 149 | 215 |

### Example 5 - In Vitro Screen in HCC1428 Breast Cancer Cells

HCC1428 human ER+ breast cancer cells were obtained from the American Type Culture Collection (ATCC) and maintained in Roswell Park Memorial Institute (RPMI) 1640 media supplemented with 10% fetal bovine serum and penicillin-streptomycin. All cells were maintained in a humidified incubator at 37° C. with 5% CO₂. 2000 cells per well were seeded into 96 well plates and allowed to incubate overnight.

The test compound was added in a matrix format in which palbociclib was added down the plate in a 5-point dose curve, diluted 4-fold starting at 2.5 µM to 9.765625 nM and respective test CDK2 inhibitor was added across the plate in an 8-point, 3-fold dilution dose curve from either 1 µM to 0.46 nM for COMPOUND C or 3 µM to 1.37 nM for COMPOUND A and COMPOUND B. Cells were incubated for 6-8 days at 37° C. with 5% CO₂. CyQuant Direct Proliferation reagent (Invitrogen) was added per manufacturer’s instructions and fluorescence was read on a Tecan M1000 plate reader. Data was analyzed with Chalice Bioinformatics Software v1.6 and ‘Synergy Score’ calculations generated where S = fcov In fX In fY Σ max(0,ldata) max(0,Idata-ILoewe), which is a positive-gated, inhibition-weighted volume over Loewe additivity. fX,Y are the dilution factors used for each single agent and the coverage factor fcov accounts for missing data, scaling the score up by the ratio of total/tested combination dose matrix points (https://horizondiscovery.com/-/media/Files/Horizon/resources/Technical-manuals/hd-technical-manual-chalice-analyzer-viewer.pdf).

Full dose response matrices and isobolograms were generated for the combinations of the test compounds and palbociclib, demonstrating the effects on proliferation of HCC1428 cells. Synergy Scores were calculated as described.

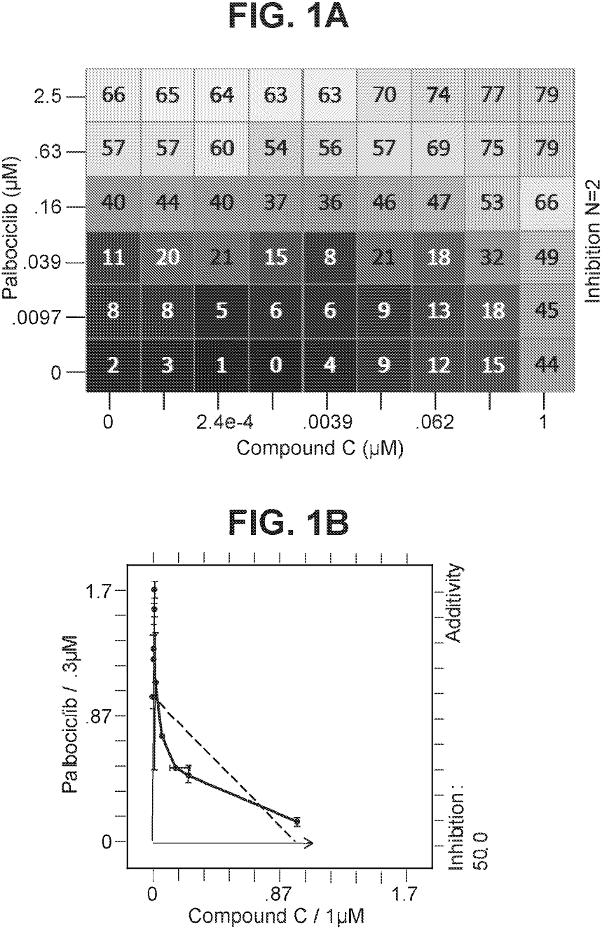

FIG. 1. shows the full dose response matrix with COMPOUND C and palbociclib for 7 days. FIG. 1A provides a heat map showing compound activity as a function of dose, where darker colors and lower numbers (bottom left) indicate no or limited activity and lighter colors and higher numbers (upper right) indicate strong activity. A Synergy Score of 1.88 was calculated. FIG. 1B provides an isobologram depicting the dose combinations at which experimental inhibition (curve) exceeded additivity (diagonal).

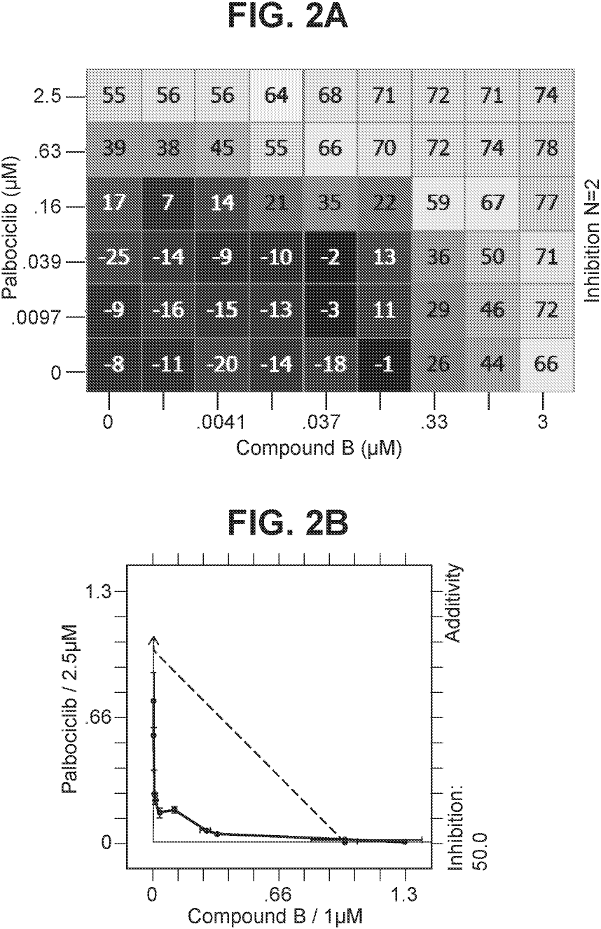

FIG. 2. shows the full dose response matrix with COMPOUND B and palbociclib for 8 days. FIG. 2A provides a heat map showing compound activity as a function of dose. A Synergy Score of 3.27 was calculated. FIG. 2B provides an isobologram depicting the dose combinations at which experimental inhibition (curve) exceeded additivity (diagonal).

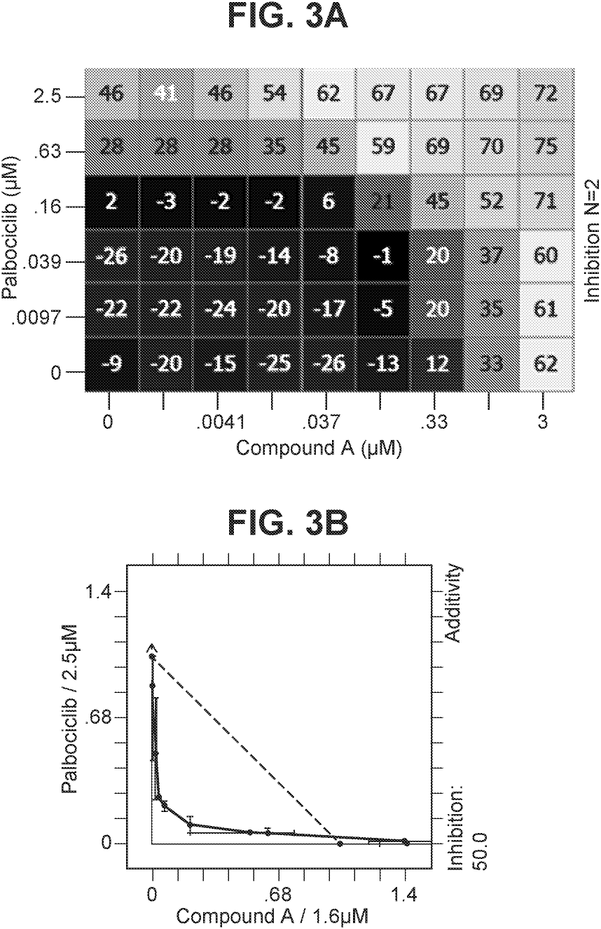

FIG. 3. shows the full dose response matrix with COMPOUND A and palbociclib for 8 days. FIG. 3A provides a heat map showing compound activity as a function of dose. A Synergy Score of 2.86 was calculated. FIG. 3B provides an isobologram depicting the dose combinations at which experimental inhibition (curve) exceeded additivity (diagonal).

### Example 6 - In Vitro Screen in MCF7 Breast Cancer Cells

MCF7 human ER+ breast cancer cells were obtained from the American Type Culture Collection (ATCC) and maintained in Roswell Park Memorial Institute (RPMI) 1640 media supplemented with 10% fetal bovine serum and penicillin-streptomycin. All cells were maintained in a humidified incubator at 37° C. with 5% CO₂. 2000 cells per well were seeded into 96 well plates and allowed to incubate overnight. Compound was added in a matrix format in which palbociclib was added down the plate in a 5-point dose curve, diluted 4-fold starting at 2.5 µM to 9.765625 nM and respective CDK2 inhibitor was added across the plate in an 8-point, 3-fold dilution dose curve from 3 µM to 1.37 nM for COMPOUND A. Cells were incubated for 6-8 days at 37° C. with 5% CO₂. CyQuant Direct Proliferation reagent (Invitrogen) was added per manufacturer’s instructions and fluorescence was read on a Tecan M1000 plate reader. Data was analyzed with Chalice Bioinformatics Software v1.6 and ‘Synergy Score’ calculations generated where S = fcov In fX In fY Σ max(0,ldata) max(0,Idata-ILoewe), which is a positive-gated, inhibition-weighted volume over Loewe additivity. fX,Y are the dilution factors used for each single agent and the coverage factor fcov accounts for missing data, scaling the score up by the ratio of total/tested combination dose matrix points (https://horizondiscovery.com/-/media/Files/Horizon/resources/Technical-manuals/hd-technical-manual-chalice-analyzer-viewer.pdf)

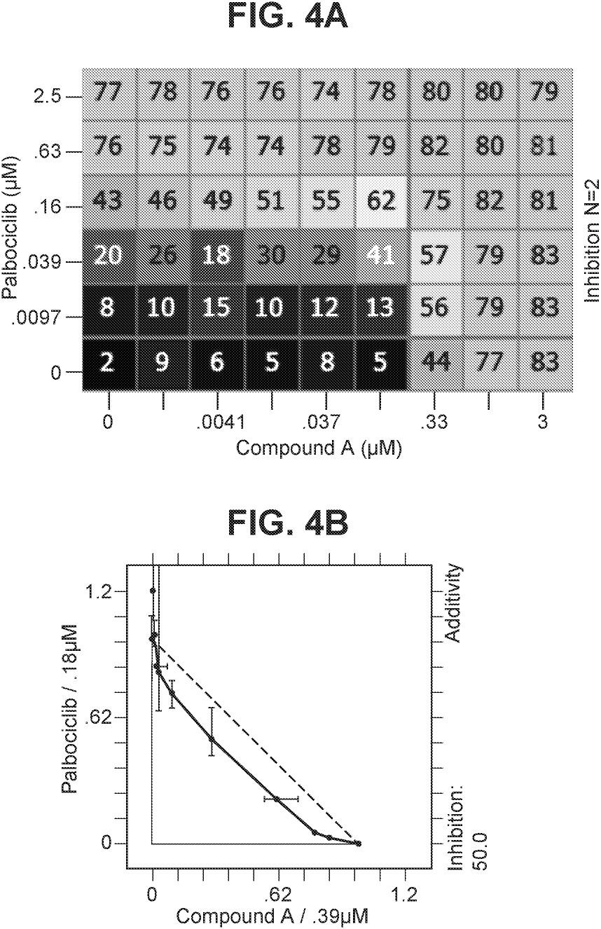

FIG. 4 shows the full dose response matrix with COMPOUND A and palbociclib for 7 days in MCF7 cells. FIG. 4A provides a heat map showing compound activity as a function of dose. A Synergy Score of 1.14 was calculated. FIG. 4B provides an isobologram depicting the dose combinations at which experimental inhibition (curve) exceeded additivity (diagonal).

### Example 7 - In Vitro Screen in T47D Breast Cancer Cells

T47D human ER+ breast cancer cells were obtained from the American Type Culture Collection (ATCC) and maintained in Roswell Park Memorial Institute (RPMI) 1640 media supplemented with 10% fetal bovine serum and penicillin-streptomycin. All cells were maintained in a humidified incubator at 37° C. with 5% CO₂. 2000 cells per well were seeded into 96 well plates and allowed to incubate overnight. Compound was added in a matrix format in which palbociclib was added down the plate in a 5-point dose curve, diluted 4-fold starting at 2.5 µM to 9.765625 nM and respective CDK2 inhibitor was added across the plate in an 8-point, 3-fold dilution dose curve from 3 µM to 1.37 nM for COMPOUND A. Cells were incubated for 6-8 days at 37° C. with 5% CO₂. CyQuant Direct Proliferation reagent (Invitrogen) was added per manufacturer’s instructions and fluorescence was read on a Tecan M1000 plate reader. Data was analyzed with Chalice Bioinformatics Software v1.6 and ‘Synergy Score’ calculations generated where S = fcov In fX In fY Σ max(0,ldata) max(0,Idata-ILoewe), which is a positive-gated, inhibition-weighted volume over Loewe additivity. fX,Y are the dilution factors used for each single agent and the coverage factor fcov accounts for missing data, scaling the score up by the ratio of total/tested combination dose matrix points (https://horizondiscovery.com/-/media/Files/Horizon/resources/Technical-manuals/hd-technical-manual-chalice-analyzer-viewer.pdf)

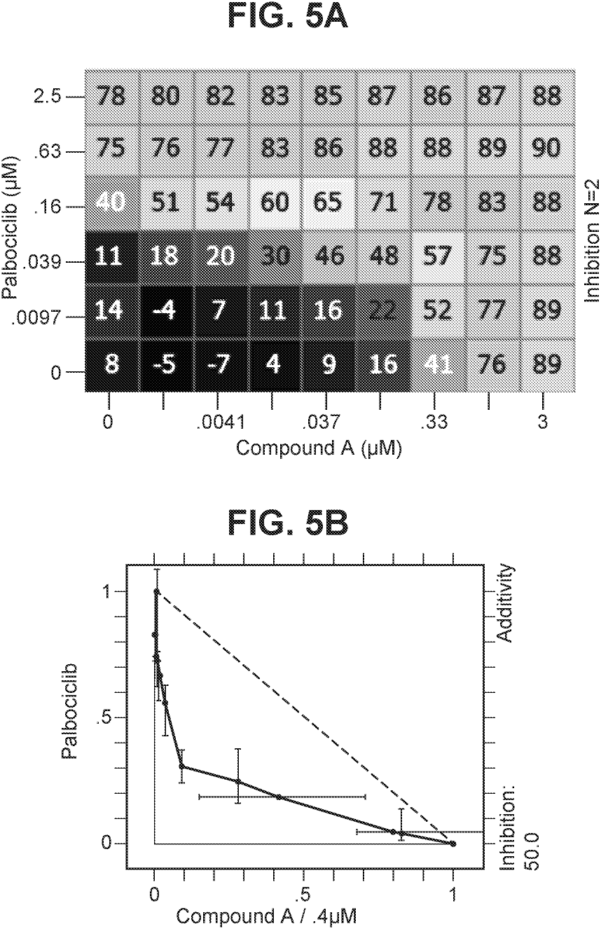

FIG. 5 shows the full dose response matrix with COMPOUND A and palbociclib for 7 days in T47D cells. FIG. 5A provides a heat map showing compound activity as a function of dose. A Synergy Score of 3.11 was calculated. FIG. 5B provides an isobologram depicting the dose combinations at which experimental inhibition (curve) exceeded additivity (diagonal).

### Example 8 - Cell Proliferation Assay

Functional effects of test compounds were measured in a 7-day anti-proliferation assay in three ER+ breast cancer cell lines. Cells were seeded in 96-well plates and allowed to adhere overnight prior to compound treatment. A top dose of 10 µM in DMSO and a 1:3 dilution dose curve in duplicate was used to determine EC₅₀ values (nM) as measured by a CyQuant Direct Cell Proliferation Assay Kit. Data are provided in Table 2 below.

TABLE 2

| | COMPOUND A | COMPOUND B | COMPOUND C | Palbociclib |
|---|---|---|---|---|
| HCC1428 | >1000 | 790 | 710 | 355 |
| MCF7 | 386 | 190 | 336 | 79 |
| T47D | 403 | 220 | 443 | 87 |

### Example 9 - In Vivo Study in MCF-7 Breast Cancer Xenograft

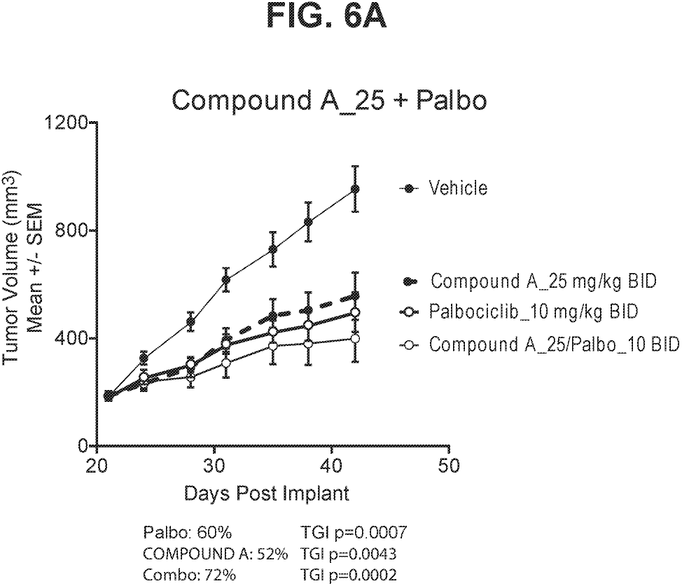

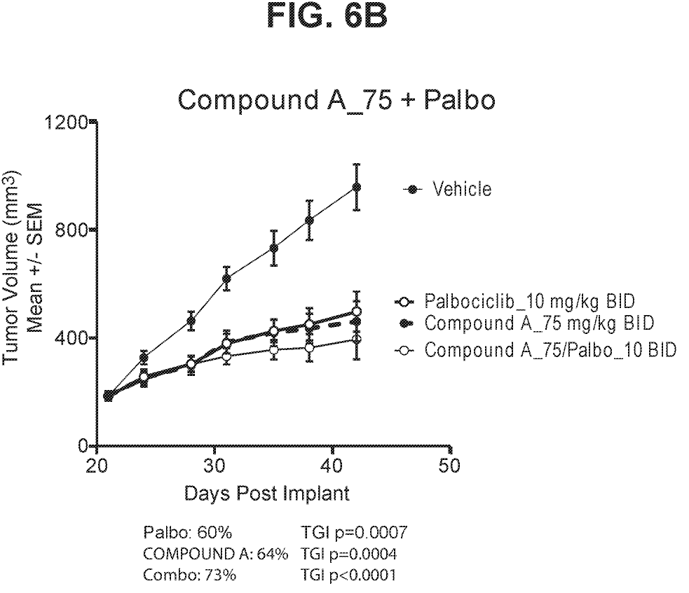

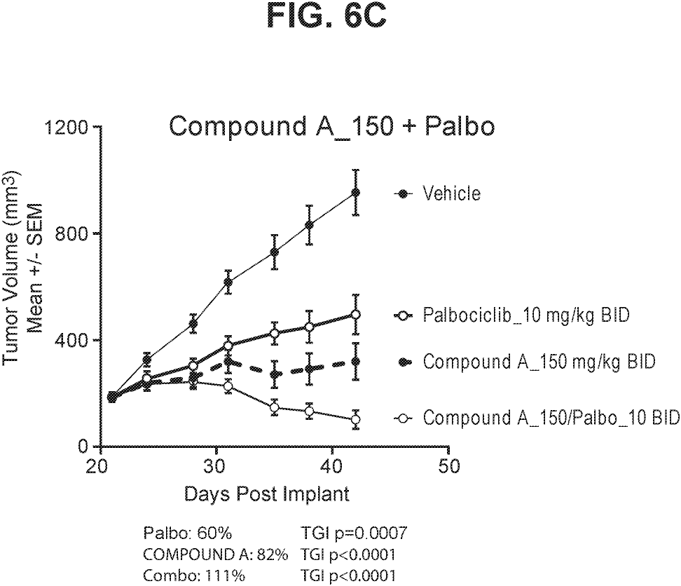

The MCF7 model was established by implanting passage 0 tumor fragments into recipient mice. To establish the MCF7 donor mice, tumor cells (5 × 106 cells/mouse with 50% Cultrex® Basement Membrane Matrix) were subcutaneously implanted in female NSG mice. Once reaching a range of 700 to 800 mm³, donor tumors were subsequently transplanted into secondary recipient mice for study expansion. When tumor volume reached a range between 100 mm³ to 291 mm³, the tumor bearing mice were randomly assigned to groups (n = 10 per group) and dosed with: 1) vehicle (0.5% MC with 0.1% Tween 80 in water); 2-4) COMPOUND A (free Base, MW = 404.22, batch No. 006) at 25, 75 and 150 mg/kg BID, Palbociclib (free Base, MW = 447.53, batch No. 039) and COMPOUND A/Palbociclib combintation respectively. 150 mg/kg COMPOUND A, po, BID is the maximum tolerated dose level in this model. COMPOUND A and Palbociclib were administered (p.o.) BID (7 hr apart). TGI was assessed on Day 21 post first dose (or Day 42 post tumor fragment implant). Tumor growth inhibition curves are provided in FIG. 6. Enhancement for the combination of palbociclib (10 mpk) and COMPOUND A at 25 mpk (FIG. 6A) or 75 mpk (FIG. 6B) was not statistically significant. Significant enhancement in tumor growth inhibition was observed for the combination of palbociclib (10 mpk) and COMPOUND A at 150 mpk (FIG. 6C). In the 150 mpk combination group, 50% of mice were found dead or were euthanized by the end of 21 days due to severe anemia.

### Example 10 - A Phase 1/2a Study of COMPOUND A as a Single Agent and in Combination With Palbociclib

A Phase 1/2a, open label, multi center, multiple dose, dose escalation, safety, PK and PD study of COMPOUND A administered as a single agent, and then in combination with a CDK4/6 inhibitor (palbociclib), and in combination with palbociclib and letrozole is conducted in selected tumor indications. The following cohorts of adult participants will be enrolled: (1) participants with advanced or metastatic SCLC, (2) participants with advanced platinum resistant ovarian cancer, (3) participants with locally recurrent, advanced or metastatic TNBC, (4) women of any menopausal status and men with HR+/HER2- advanced or metastatic breast cancer, and (5) participants with advanced or metastatic NSCLC.

Dose escalation is conducted in Part 1A to determine the maximum tolerated dose (MTD) and recommended Phase 2 dose (RP2D) for COMPOUND A as a single agent in participants with advanced or metastatic SCLC, advanced platinum resistant ovarian cancer, locally recurrent/advanced or metastatic TNBC, women of any menopausal status and men with HR+/HER2- advanced or metastatic breast cancer, and in advanced or metastatic NSCLC.

Dose finding with COMPOUND A in combination with palbociclib, and COMPOUND A in combination with palbociclib and letrozole is conducted in Part 1B. Part 1B will include participants with advanced platinum resistant epithelial ovarian cancer (EOC), primary peritoneal carcinomatosis (PPC), and fallopian tube cancer (FTC), locally recurrent/advanced or metastatic TNBC, and women of any menopausal status and men with HR+/HER2- advanced or metastatic breast cancer.

Dose expansion is conducted in Part 2 and will include COMPOUND A as a single agent, in combination with palbociclib, and in combination with palbociclib and letrozole, in dose expansion arms with SCLC, ovarian cancer, TNBC, and HR-positive HER2-negative advanced or metastatic breast cancer.

A Bayes logistic regression model (BLRM) along with the escalation with overdose controls (EWOC) criteria will be used in Parts 1A and 1B to guide the dose escalation and to determine the MTD/RP2D of COMPOUND A as monotherapy and in combination with palbociclib, or in combination with palbociclib and letrozole.

All publications and patent applications cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

1. A method of treating cancer in a subject in need thereof comprising administering to the subject:
(a) an amount of a compound of Formula (I): 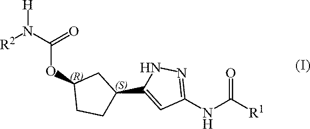
or a pharmaceutically acceptable salt thereof, wherein:
R¹ is —L—(5-6 membered heteroaryl) or —L—(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) an amount of a cyclin dependent kinase 4/6 (CDK4/6) inhibitor;
wherein the amounts in (a) and (b) together are effective in treating cancer.

2. The method of claim 1, further comprising administering to the subject: (c) an amount of an additional anti-cancer agent; wherein the amounts in (a), (b) and (c) together are effective in treating cancer.

3. The method of claim 1 or 2, wherein the compound of Formula (I) is selected from the group consisting of:
(1R,3S)-3-[3-({[3-(methoxymethyl)-1-methyl-1H-pyrazol-5-yl]carbonyl}amino)-1H-pyrazol-5-yl]cyclopentyl propan-2-ylcarbamate;
(1R,3S)-3-[3-({[2-(methylsulfonyl)phenyl]acetyl}amino)-1H-pyrazol-5-yl]cyclopentyl (2S)-butan-2-ylcarbamate; and
(1R,3S)-3-(3-{[(2-methoxypyridin-4-yl)acetyl]amino}-1H-pyrazol-5-yl)cyclopentyl propylcarbamate;
or a pharmaceutically acceptable salt thereof.

4. The method of any one of claims 1 to 3, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate.

5. The method of any one of claims 1 to 4, wherein the CDK4/6 inhibitor is palbociclib, or a pharmaceutically acceptable salt thereof.

6. The method of any one of claims 1 to 5, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

7. The method of any one of claims 2 to 6, wherein the cancer is hormone receptor positive (HR+), human epidermal growth factor receptor 2 negative (HER2-) breast cancer and the additional anti-cancer agent is an endocrine therapeutic agent selected from the group consisting of an aromatase inhibitor, a SERM and a SERD.

8. The method of claim 7, wherein the endocrine therapeutic agent is letrozole or fulvestrant.

9. A combination comprising:
(a) a compound of Formula (I): 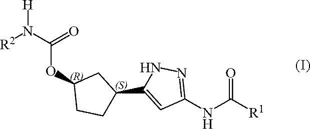
or a pharmaceutically acceptable salt thereof, wherein:
R¹ is —L—(5-6 membered heteroaryl) or —L—(phenyl), where said 5-6 membered heteroaryl or phenyl is optionally substituted by one to three R³;
R² is C₁-C₆ alkyl or C₃-C₇ cycloalkyl, where said C₃-C₇ cycloalkyl is optionally substituted by C₁-C₄ alkyl;
L is a bond or methylene; and
each R³ is independently C₁-C₄ alkyl, C₁-C₄ alkoxy or SO₂-C₁-C₄ alkyl, where each C₁-C₄ alkyl is optionally substituted by F, OH or C₁-C₄ alkoxy; and
(b) a cyclin dependent kinase 4/6 (CDK4/6) inhibitor;
wherein the combination of (a) and (b) is effective in treating cancer.

10. The combination of claim 9, further comprising (c) an additional anti-cancer agent; wherein the combination of (a), (b) and (c) is effective in treating cancer.

11. The combination of claim 9 or 10, wherein the compound of Formula (I) is selected from the group consisting of:
(1R,3S)-3-[3-({[3-(methoxymethyl)-1-methyl-1H-pyrazol-5-yl]carbonyl}amino)-1H-pyrazol-5-yl]cyclopentyl propan-2-ylcarbamate;
(1R,3S)-3-[3-({[2-(methylsulfonyl)phenyl]acetyl}amino)-1H-pyrazol-5-yl]cyclopentyl (2S)-butan-2-ylcarbamate; and
(1R,3S)-3-(3-{[(2-methoxypyridin-4-yl)acetyl]amino}-1H-pyrazol-5-yl)cyclopentyl propylcarbamate;
or a pharmaceutically acceptable salt thereof.

12. The combination of any one of claims 9 to 11, wherein the compound of Formula (I) is (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate.

13. The combination of any one of claims 9 to 12, wherein the CDK4/6 inhibitor is palbociclib, or a pharmaceutically acceptable salt thereof.

14. The combination of any one of claims 9 to 13, wherein the cancer is selected from the group consisting of breast cancer, lung cancer, ovarian cancer, peritoneal cancer, fallopian tube cancer, bladder cancer, colon cancer, uterine cancer, prostate cancer, esophageal cancer, liver cancer, pancreatic cancer and stomach cancer.

15. The combination of any one of claims 9 to 14, wherein the cancer is HR+, HER2- breast cancer and the additional anti-cancer agent is an endocrine therapeutic agent selected from the group consisting of an aromatase inhibitor, a SERM and a SERD.

16. The combination of claim 15, wherein the endocrine therapeutic agent is letrozole or fulvestrant.

17. A method of treating cancer in a subject in need thereof comprising administering to the subject:
(a) an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate; and
(b) an amount of palbociclib, or a pharmaceutically acceptable salt thereof;
wherein the amounts in (a) and (b) together are effective in treating cancer.

18. A method of treating cancer in a subject in need thereof comprising administering to the subject:
(a) an amount of (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate;
(b) an amount of palbociclib, or a pharmaceutically acceptable salt thereof; and
(c) an amount of an endocrine therapeutic agent selected from the group consisting of an aromatase inhibitor, a SERM and a SERD;
wherein the amounts in (a), (b) and (c) together are effective in treating cancer.

19. A combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate; and
(b) palbociclib, or a pharmaceutically acceptable salt thereof;
wherein the combination of (a) and (b) is effective in treating cancer.

20. A combination comprising:
(a) (1R,3S)-3-[3-({[3-(methoxymethyl)-1-methyl-1H-pyrazol-5-yl]carbonyl}amino)-1H-pyrazol-5-yl]cyclopentyl propan-2-ylcarbamate;
(b) palbociclib, or a pharmaceutically acceptable salt thereof; and
(c) an endocrine therapeutic agent selected from the group consisting of an aromatase inhibitor, a SERM and a SERD;
wherein the combination of (a), (b) and (c) is effective in treating cancer.


SEQUENCE LISTING (2 sequences)

SEQ ID NO: 1 - protein, 6 aa, Artificial
  Synthetic Construct
  MISC_FEATURE (1)..(1): 5-FAM labeled Glutamine
  MISC_FEATURE (6)..(6): Glycine carboxamide
QSPKKG

SEQ ID NO: 2 - protein, 15 aa, Artificial
  Synthetic Construct
  MISC_FEATURE (1)..(1): 5-FAM labeled Arginine
RRRFRPASPLRGPPK